(19) United States
(12) Patent Application Publication
(10) Pub. No.: US 2024/0416075 A1
(43) Pub. Date: Dec. 19, 2024

(54) MEDICAL SYSTEM INCLUDING CATHETER APPARATUS

(71) Applicant: CANON KABUSHIKI KAISHA, Tokyo (JP)

(72) Inventors: KEIGO AKIYA, Tokyo (JP); KEI SATO, Kanagawa (JP)

(21) Appl. No.: 18/815,009

(22) Filed: Aug. 26, 2024

Related U.S. Application Data
(63) Continuation of application No. PCT/JP2023/006438, filed on Feb. 22, 2023.

(30) Foreign Application Priority Data
Feb. 28, 2022 (JP) 2022-028903
Jan. 31, 2023 (JP) 2023-012442

(51) Int. Cl. A61M 25/01

(52) U.S. Cl. CPC A61M 25/01; A61M 2205/3306; A61M 2205/3327; A61M 2205/502; A61M 2205/583

(57) ABSTRACT

A medical system includes a catheter apparatus including a bendable member having a distal end, a receiving section to which the catheter apparatus is attached, a support base that supports the receiving section to move linearly between a forward position and a retracted position, a cart connected to the support base, the cart being configured to move the catheter apparatus, and a display configured to display reach information related to a position of the distal end.

### CROSS-REFERENCE TO RELATED APPLICATIONS

This application is a Continuation of International Patent Application No. PCT/JP2023/006438, filed Feb. 22, 2023, which claims the benefit of Japanese Patent Application No. 2022-028903, filed Feb. 28, 2022, and Japanese Patent Application No. 2023-012442, filed Jan. 31, 2023, all of which are hereby incorporated by reference herein in their entireties.

### TECHNICAL FIELD

The present invention relates to a medical system including a catheter apparatus to be inserted into a patient's lumen.

### BACKGROUND ART

Surgical planning using two-dimensional or three-dimensional image data (model data) has recently become popular in medical front. Surgery using a catheter is also included as one of the subjects of surgical planning. Medical robots have also come to be used in inserting a catheter through a lumen entry such as the patient's mouse or nose to perform observation and examinations inside the patient's body using instruments such as endoscopes.

In a manipulator support structure (medical robot) disclosed in PTL 1, a catheter manipulator assembly including a catheter cartridge is attached to a support frame with a support connector corresponding to an arm. The manipulator support structure (medical robot) disclosed in PTL 1 also includes multiple wheels to enable the user (a doctor or assistant) to move the medical robot to a work area as occasion arises. In the work area after the movement, the user moves the medical robot to determine a position relative to the patient so that the distal end of the catheter is located in the vicinity of the lumen entry of the patient and thereafter operates the catheter cartridge to perform surgery or biopsy.

### CITATION LIST

### Patent Literature

PTL 1 Japanese Patent Laid-Open No. 2014-158942

However, the positioning of medical robots (medical systems) relative to patients has conventionally been performed based on the user's experience. For this reason, if the relative position of the medical robot and the patient is not proper, the distal end of the catheter may not reach the initial target position. This may need to interrupt the examination, etc. to correct the positions of the medical robot and the patient. This may lead to an increase in the total amount of work and time for observation or examination, resulting in an increase in the load on the user and the patient.

### SUMMARY OF INVENTION

The present invention provides a mechanism for supporting the positioning of a medical system relative to a patient.

An embodiment of the present invention is a medical system including a catheter apparatus including a bendable member having a distal end, a receiving section to which the catheter apparatus is attached, a support base that supports the receiving section to move linearly between a forward position and a retracted position, a cart connected to the support base, the cart being configured to move the catheter apparatus, and a display configured to display reach information related to a position of the distal end.

Further features of the present invention will become apparent from the following description of exemplary embodiments with reference to the attached drawings.

### BRIEF DESCRIPTION OF DRAWINGS

FIG. 1A is an overall perspective view of a medical system.

FIG. 1B is a control block diagram of the medical system.

FIG. 2 is an explanatory diagram illustrating an example of positioning of the medical system relative to the patient.

FIG. 3 is a perspective view of a catheter apparatus and a support base.

FIG. 4A is a diagram illustrating a state in which a catheter is inserted into the patient's oral cavity.

FIG. 4B is a diagram illustrating a state in which the catheter is inserted into the patient's oral cavity.

FIG. 5A is a diagram illustrating the entire catheter.

FIG. 5B is an enlarged view of the catheter.

FIG. 6 is an explanatory diagram of a catheter unit.

FIG. 7A is a perspective view of a base unit and a wire driving section.

FIG. 7B is a side view of the base unit and the wire driving section.

FIG. 7C is a front view of the base unit and the wire driving section.

FIG. 8A is a perspective view of the wire driving section, the connector, and the bend driving section.

FIG. 8B is a perspective view of the wire driving section, the connector, and the bend driving section.

FIG. 8C is a perspective view of the wire driving section, the connector, and the bend driving section.

FIG. 9 is a perspective view of the catheter apparatus illustrating coordinate setting in the moving direction of the catheter apparatus and the stage.

FIG. 10A is a diagram illustrating the movable range of the catheter apparatus supported by the support base.

FIG. 10B is a diagram illustrating the movable range of the catheter apparatus supported by the support base.

FIG. 10C is a diagram illustrating the movable range of the catheter apparatus supported by the support base.

FIG. 10D is a diagram illustrating the movable range of the catheter apparatus supported by the support base.

FIG. 11A is a diagram illustrating the movable range of the catheter apparatus due to the movement of the arm and the support shaft.

FIG. 11B is a diagram illustrating the movable range of the catheter apparatus due to the movement of the arm and the support shaft.

FIG. 12 is a diagram illustrating an example of medical image data.

FIG. 13 is a diagram illustrating an example of a catheter apparatus installation navigation chart.

FIG. 14A is a side view of a support base equipped with a linear member and the catheter apparatus attached to the support base.

FIG. 14B is a side view of the support base equipped with the linear member and the catheter apparatus attached to the support base.

FIG. 15 is a perspective view of the support base equipped with the linear member and the catheter apparatus attached to the support base.

FIG. 16A is a diagram illustrating an example of the linear member.

FIG. 16B is a diagram illustrating another example of the linear member.

FIG. 16C is a diagram illustrating still another example of the linear member.

FIG. 17A is another side view of a support base equipped with a linear member and the catheter apparatus attached to the support base.

FIG. 17B is another side view of the support base equipped with the linear member and the catheter apparatus attached to the support base.

FIG. 18A is another side view of the support base equipped with the linear member and the catheter apparatus attached to the support base.

FIG. 18B is another side view of the support base equipped with the linear member and the catheter apparatus attached to the support base.

FIG. 19 is a perspective view of the support base including a measurement sensor for confirming the distal end position of the catheter.

FIG. 20 is a diagram illustrating an example of mouthpiece placement on the patient.

FIG. 21A is a diagram illustrating measurement using a measurement sensor.

FIG. 21B is a diagram illustrating measurement using a measurement sensor.

### DESCRIPTION OF EMBODIMENTS

Embodiments of the present invention will be described with reference to the drawings. It is to be understood that the dimensions, materials, shapes, and positions of the components described in the embodiments may be appropriately changed according to the configurations and various conditions of the apparatuses to which the present invention is applied.

### First Embodiment

### Medical System and Medical Apparatus

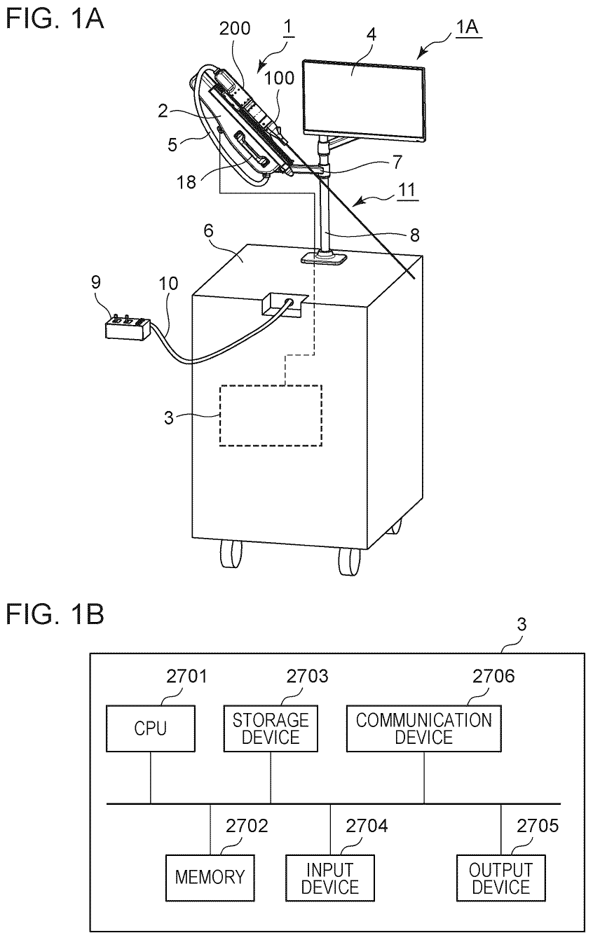

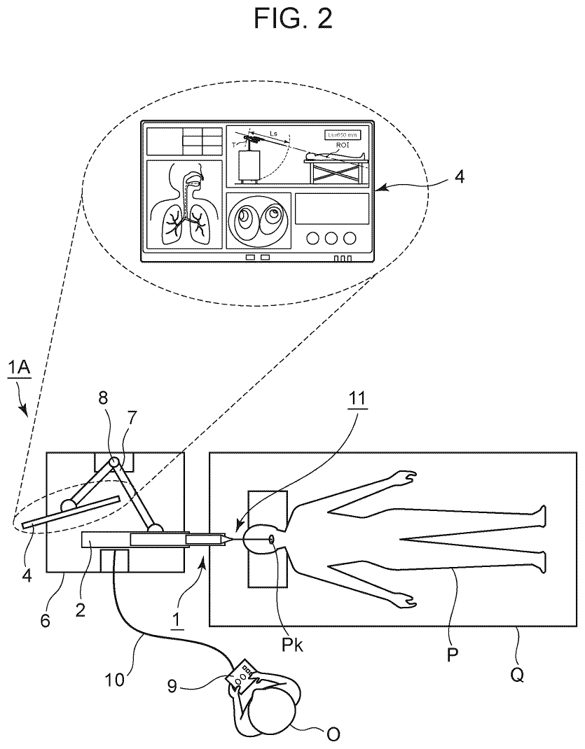

FIG. 1A is an overall perspective view of a medical system. FIG. 1B is a control block diagram. FIG. 2 is an explanatory diagram illustrating an example of positioning of the medical system relative to a patient.

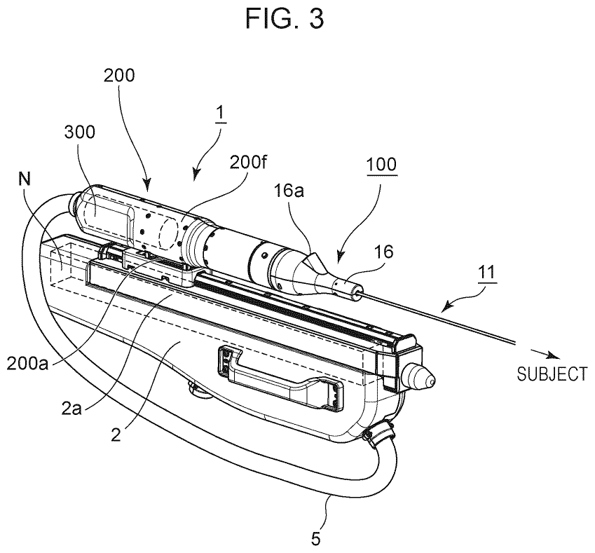

A medical system 1A includes a catheter apparatus 1, a stage (a receiving section) 2a (see FIG. 3), a support base 2 that supports the stage 2a to allow linear movement, an arm 7 connected to the support base 2 to allow the support base 2 to move, and a support shaft 8 connected to the arm 7. In other words, the support base 2 supports the catheter apparatus 1 to allow linear movement. A connection between the arm 7 and the support base 2 is located at one end of the arm 7, and a connection between the arm 7 and the support shaft 8 is located at the other end of the arm 7. The medical system 1A includes a cart 6 connected to the support shaft 8 to movably install the catheter apparatus 1 and a controller 3 for controlling the catheter apparatus 1. In other words, the cart 6 is connected to the support base 2 via the support shaft 8 and the arm 7 to support the support base 2. In other words, the cart 6 is connected to the stage 2a and the catheter apparatus 1 via the support base 2 to support the stage 2a and the catheter apparatus 1. The medical system 1A includes a monitor 4 serving as a display.

First, FIG. 1A will be described. The catheter apparatus 1 includes a catheter unit 100 including a catheter 11 (a bendable member) and a base unit 200.

In this embodiment, the user of the medical system 1A and the catheter apparatus 1 locates the catheter apparatus 1 and the subject at a predetermined position so as to insert the catheter 11 into the subject. The user here refers to a healthcare professional such as a doctor or his/her assistant.

In one embodiment, the user places the patient on his/her back on an operating table Q. The user moves the medical system 1A to the vicinity of the patient using the cart 6 having casters on the bottom. The user adjusts the orientations and angles of the support base 2, the arm 7, the cart 6, and so on and locate them so that the catheter apparatus 1 moves straight ahead to the entry of the oral cavity or nasal cavity of the patient. The support base 2 and the arm 7 can easily be adjusted by gripping a handle 18 provided on the support base 2. The user can perform tasks such as observing the interior of the disposed subject, collecting various samples from inside the subject, and conducting procedures inside the subject by inserting the catheter 11 into the subject. In one embodiment, the user may insert the catheter 11 into the patient's lumen. Specifically, the user may perform tasks such as observing, collecting, and ablating lung tissue by inserting the catheter 11 into the bronchial tube of the patient through the oral cavity or nasal cavity.

The catheter 11 may be used as a guide (a sheath) for guiding a medical device for conducting the above tasks. Examples of the medical device (tool) include an endoscope, forceps, and an ablation device. The catheter 11 itself may have the functions as the medical devices.

The controller 3 is an example of an information processing apparatus incorporating the present invention. FIG. 1B illustrates the details of the controller 3. FIG. 1B is a control block diagram of the controller 3. The controller 3 includes a central processing unit (CPU) 2701, a memory 2702, a storage device 2703, an input device 2704, an output device 2705, and a communication device 2706. The CPU 2701 is responsible for various controls in the medical system 1A, such as executing various calculations (described below), controlling the catheter apparatus 1, and displaying a variety of information on the monitor 4, by reading and executing programs stored in the storage device 2703. The memory 2702 functions as a volatile storage and serves as a working area for the CPU 2701 to perform various computations. The storage device 2703 functions as a non-volatile storage and stores various programs, various parameters, various display information, and patient data. The input device 2704 receives commands and inputs for operating the catheter 11 from an operating unit 9 (described below). The input device 2704 inputs data input via a keyboard (not shown) or the communication device 2706 to the storage device 2703 of the controller 3. If the monitor 4 functions as a touch panel, the data input via the touch panel to the storage device 2703 of the controller 3. The output device 2705 outputs signals for displaying images on the monitor 4 that is electrically connected to the controller 3, according to an instruction from the CPU 2701. The monitor 4 that has received the display information displays information for operating the medical system 1A on the monitor screen.

Referring back to FIG. 1A, the operating unit 9 issues commands to move the stage 2a to insert the catheter 11 into the subject, to bend the catheter 11, and to use the medical system 1A. The operating unit 9 includes a joystick, a switch, a push button, and the like to operate the control target. The operating unit 9 may include a foot switch (not shown) on the installation surface of the medical system 1A. The operating unit 9 is electrically connected to the controller 3 via a cable 10 and the cart 6. The operating unit 9 and the controller 3 may be directly connected by the cable 10 or may be connected via the support base 2. The operating unit 9 may be integrated with the cart 6 or the support base 2 as part of the medical system 1A. The transmission and reception of information between the operating unit 9 and the controller 3 does not have to be via wired connection and may be via wireless connection using various communication methods. The operating unit 9 and the controller 3 may be connected by radio.

### Details of Catheter Apparatus 1 and Support Base 2

FIG. 3 is a perspective view of the catheter apparatus 1 and the support base 2. As illustrated in FIG. 3, the catheter apparatus 1 in this embodiment is electrically connected to the controller 3 via a cable 5 that connects a base unit 200 of the catheter apparatus 1 to the support base 2, and via the support base 2. The catheter apparatus 1 and the controller 3 may be directly connected by a cable. The catheter apparatus 1 and the controller 3 may be connected by radio.

The catheter apparatus 1 is detachably attached to the support base 2 via the base unit 200. More specifically, an attaching section 200a of the base unit 200 of the catheter apparatus 1 is detachably attached to the stage 2a supported by the support base 2. The catheter apparatus 1 is attachable to multiple positions on the stage 2a via the attaching section 200a. The connection between the catheter apparatus 1 and the controller 3 is maintained so as to be able to control the catheter apparatus 1 with the controller 3 even if the attaching section 200a of the catheter apparatus 1 is detached from the stage 2a. In this embodiment, the catheter apparatus 1 and the support base 2 are connected by the cable 5 even if the attaching section 200a of the catheter apparatus 1 is detached from the stage 2a.

In one embodiment, the user moves the catheter apparatus 1 manually to insert the catheter 11 into the branch point of the bronchial tubes of the patient through the oral cavity or the nasal cavity, with the catheter apparatus 1 detached from the support base 2 (with the (catheter apparatus 1 detached from the stage 2a).

The user inserts the catheter 11 into the bronchial tube and thereafter reattaches the catheter apparatus 1 to a desired position among the multiple attachment positions on the stage 2a. After the catheter apparatus 1 is reattached, the user can operate the catheter apparatus 1. Specifically, the stage 2a is configured to move rectilinearly relative to the support base 2. As the stage 2a moves, with the catheter apparatus 1 attached to the stage 2a, the catheter apparatus 1 moves in a straight line relative to the support base 2. The rectilinear movement includes forward movement and backward movement. When the stage 2a moves forward, the catheter 11 moves toward the target position. When the stage 2a moves backward, the catheter 11 moves in the drawn-out direction. The movement of the stage 2a is based on a control instruction from the controller 3.

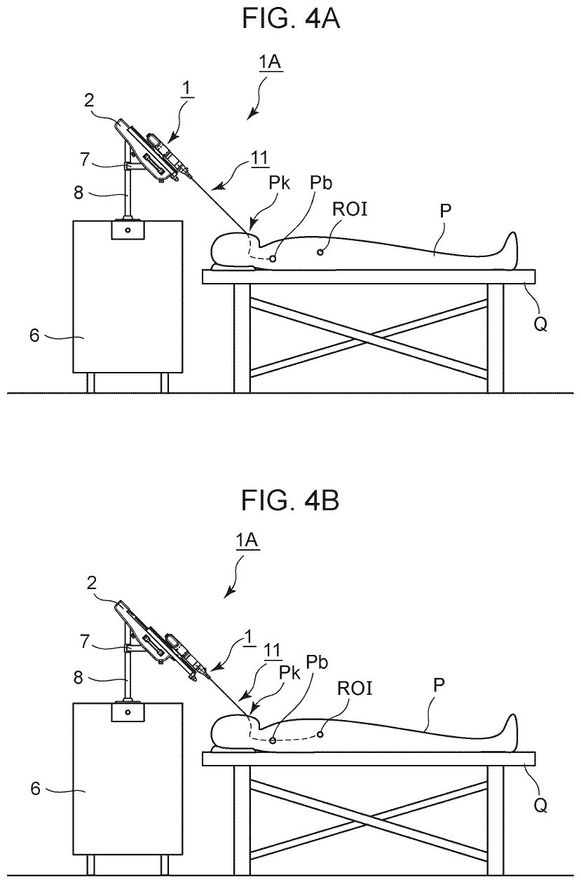

FIG. 4A illustrates a state in which the catheter apparatus 1 is manually moved by the user, and the catheter 11 is inserted to the branch point Pb of the bronchial tubes of a patient P who lies on his/her back on the operating table Q via the mouth Pk and the oral cavity or the nasal cavity. The region of interest (ROI) (described below) corresponds to the affected part or biopsy part of the patient P.

FIG. 4B illustrates a state in which the distal end (the leading end) of the catheter 11 is moved to the ROI by the user operating the operating unit 9 to move the stage 2a from the state in FIG. 4A.

The support base 2 serves both as a support for supporting the catheter apparatus 1 and a linear motion mechanism for moving the catheter apparatus 1 in the insertion direction and in the drawing-out direction. The support base 2 includes a stage drive unit N for driving the stage 2a. The stage drive unit N operates according to a control instruction from the controller 3. More specifically, the control instruction from the controller 3 is based on an operation from the operating unit 9. The stage drive unit N includes an output shaft (a motor shaft), a motor for rotating the output shaft clockwise (CW) and counterclockwise (CCW), and a drive conversion section connected to the output shaft to convert the rotation of the output shaft to translatory movement in the direction in which the stage 2a moves forward or backward. Examples of the drive conversion section include a feed screw, a ball screw, a rack-and-pinion mechanism, a slider-crank mechanism, and other various configurations.

In this embodiment, the motor is a stepping motor. When the output shaft is rotated clockwise (CW) by the operation of the motor, the rotation is converted to a linear motion by the drive conversion section, and the stage 2a moves in the direction in which the catheter 11 is inserted into the subject. When the output shaft is rotated counterclockwise (CCW) by the operation of the motor, the rotation is converted to a linear motion by the drive conversion section, and the stage 2a moves in the direction in which the catheter 11 is drawn out of the subject.

In another embodiment, the attaching section 200a of the base unit 200 includes a canceling switch and a release switch (not shown). The user can move the catheter apparatus 1 manually along the guide direction of the stage 2a while pressing the canceling switch, with the attaching section 200a attached to the stage 2a. In other words, the stage 2a includes a guide configuration for guiding the movement of the catheter apparatus 1. When the user stops pressing the canceling switch, the catheter apparatus 1 is fixed to the stage 2a. In contrast, when the user presses the release switch, with the attaching section 200a attached to the stage 2a, the user can detach the catheter apparatus 1 from the stage 2a. Moving the catheter apparatus 1 guided by the guide configuration of the stage 2a allows the user to move the catheter apparatus 1 to a desired position among multiple attaching positions on the stage 2a and to attach the catheter apparatus 1 to the desired position.

A single switch may have both of the functions of the canceling switch and the release switch. Providing a mechanism for switching between pressing and non-pressing states for the canceling switch eliminates the need for user to continue to press the canceling switch when manually sliding the catheter apparatus 1.

The catheter apparatus 1 is fixed to the stage 2a and moved by the stage 2a driven by the motor (not shown), with the attaching section 200a is attached to the stage 2a, and the canceling switch and the release switch not pressed.

The catheter apparatus 1 includes a wire driving section (a linear-member driving section, a line driving section, or a main-body driving section) 300 for driving the catheter 11. In this embodiment, the catheter apparatus 1 is a robot catheter apparatus that drives the catheter 11 using the wire driving section 300 controlled by the controller 3.

The controller 3 may control the wire driving section 300 to bend the catheter 11. In this embodiment, the wire driving section 300 is housed in the base unit 200. More specifically, the base unit 200 includes a base casing 200f that houses the wire driving section 300. In other words, the base unit 200 includes the wire driving section 300. A combination of the wire driving section 300 and the base unit 200 may be referred to as a catheter driving unit (a base unit or a main body).

The leading end of the catheter 11 to be inserted into the subject in the extending direction of the catheter 11 is referred to as a distal end. The end of the catheter 11 opposite to the distal end in the extending direction of the catheter 11 is referred to as a proximal end.

The catheter unit 100 includes a proximal end cover 16 covering the proximal end of the catheter 11. The proximal end cover 16 includes a tool hole 16a. A medical device may be inserted into the catheter 11 through the tool hole 16a.

In this embodiment, the catheter 11 has a function as a guide for guiding a medical device to a desired position in the subject, as described above.

For example, the catheter 11 is inserted to a target position in the subject, with an endoscope inserted in the catheter 11. In this case, at least one of a user's manual operation, movement of the stage 2a, and driving with the wire driving section 300 is used to move the catheter 11. After the catheter 11 reaches the target position, the endoscope is drawn out of the catheter 11 through the tool hole 16a. Then, a medical device is inserted through the tool hole 16a to perform tasks such as collecting various samples and procedures for the interior of the subject.

For example, the medical system 1A with such a configuration allows for displaying images captured by an endoscope inserted into the catheter 11 on the monitor 4 by connecting the endoscope to the monitor 4 Connecting the monitor 4 and the controller 3 allows for displaying information related to the state of the catheter apparatus 1 and control of the catheter apparatus 1 on the monitor 4. For example, information related to the position of the catheter 11 in the subject and information related to the navigation of the catheter 11 in the subject may be displayed on the monitor 4. The monitor 4, the controller 3, and the endoscope may be connected by wire or wirelessly. The monitor 4 and the controller 3 may be connected via the support base 2.

### Catheter

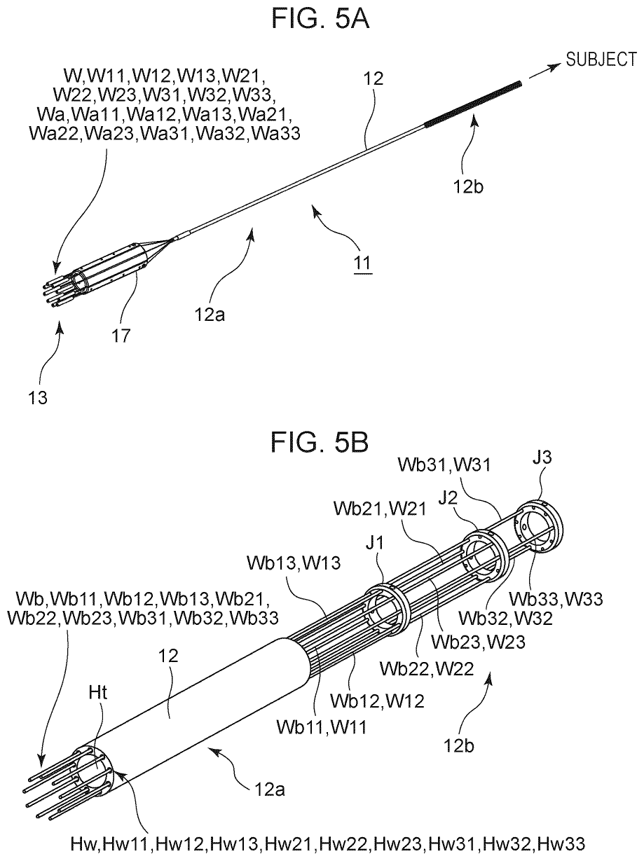

Referring to FIGS. 5A and 5B, the catheter 11 (a bendable member) will be described. FIGS. 5A and 5B are explanatory diagram of the catheter 11. FIG. 5A is a diagram illustrating the entire catheter 11. FIG. 5B is an enlarged view of the catheter 11. The catheter 11 includes a bendable section (a bendable body or a catheter main body) 12 and a bend driving section (a catheter driving section) 13 configured to bend the bendable section 12. The bend driving section 13 is configured to bend the bendable section 12 using the driving force of the wire driving section 300 received via a connector 21 (described below).

The catheter 11 extends along the direction in which the catheter 11 is to be inserted into the subject. The extending direction (longitudinal direction) of the catheter 11 is the same as the extending direction (longitudinal direction) of the bendable section 12 and the extending direction (longitudinal direction) of first to ninth drive wires (W11 to W33) described below.

The bend driving section 13 includes multiple drive wires (drive lines, linear members, or linear actuators) connected to the bendable section 12. Specifically, the bend driving section 13 includes the first drive wire W11, the second drive wire W12, the third drive wire W13, the fourth drive wire W21, the fifth drive wire W22, the sixth drive wire W23, the seventh drive wire W31, the eighth drive wire W32, and the ninth drive wire W33.

Each of the first to ninth drive wires (W11 to W33) includes a retained portion (a retained shaft or a rod) Wa. Specifically, the first drive wire W11 includes a first retained portion Wa11. The second drive wire W12 includes a second retained portion Wa12. The third drive wire W13 includes a third retained portion Wa13. The fourth drive wire W21 includes a fourth retained portion Wa21. The fifth drive wire W22 includes a fifth retained portion Wa22. The sixth drive wire W23 includes a sixth retained portion Wa23. The seventh drive wire W31 includes a seventh retained portion Wa31. The eighth drive wire W32 includes an eighth retained portion Wa32. The ninth drive wire W33 includes a ninth retained portion Wa33.

In this embodiment, the first to ninth retained portions (Wa11 to Wa33) have the same shape.

Each of the first to ninth drive wires (W11 to W33) includes a flexible wire body (a line body or a linear body) Wb. Specifically, the first drive wire W11 includes a first wire body Wb11. The second drive wire W12 includes a second wire body Wb12. The third drive wire W13 includes a third wire body Wb13. The fourth drive wire W21 includes a fourth wire body Wb21. The fifth drive wire W22 includes a fifth wire body Wb22. sixth drive wire W23 includes a sixth wire body Wb23. The seventh drive wire W31 includes a seventh wire body Wb31. The eighth drive wire W32 includes an eighth wire body Wb32. The ninth drive wire W33 includes a ninth wire body Wb33.

In this embodiment, the first to third wire bodies (Wb11 to Wb13) have the same shape. The fourth to sixth wire bodies (Wb21 to Wb23) have the same shape. The seventh to ninth wire bodies (Wb31 to Wb33) have the same shape. In this embodiment, the first to ninth wire bodies (Wb11 to Wb33) have the same shape except the length.

The first to ninth retained portions (Wa11 to Wa33) are fixed to the first to ninth wire bodies (Wb11 to Wb33) at the proximal ends of the first to ninth wire bodies (Wb11 to Wb33), respectively.

The first to ninth drive wires (W11 to W33) are inserted into the bendable section 12 via a wire guide 17 and are fixed therein.

In this embodiment, the material of the first to ninth drive wires (W11 to W33) is metal. The material of the first to ninth drive wires (W11 to W33) may be resin. The material of the first to ninth drive wires (W11 to W33) may contain metal and resin.

Any one of the first to ninth drive wires (W11 to W33) may be referred to as a drive wire W. In this embodiment, the first to ninth drive wires (W11 to W33) have the same shape except the lengths of the first to ninth wire bodies (Wb11 to Wb33).

In this embodiment, the bendable section 12 is a flexible tubular member including a passage Ht for use in inserting a medical device.

The wall of the bendable section 12 has multiple wire holes for passing the first to ninth drive wires (W11 to W33) individually. Specifically, the wall of the bendable section 12 has a first wire hole Hw11, a second wire hole Hw12, a third wire hole Hw13, a fourth wire hole Hw21, a fifth wire hole Hw22, a sixth wire hole Hw23, a seventh wire hole Hw31, an eighth wire hole Hw32, and a ninth wire hole Hw33. The first to ninth wire holes Hw (Hw11 to Hw33) correspond to the first to ninth drive wires (W11 to W33), respectively. The numerals following the sign Hw indicate the corresponding drive wires. For example, the first drive wire W11 is inserted into the first wire hole Hw11.

Any one of the first to ninth wire holes (Hw11 to Hw33) may be referred to as a wire hole Hw. In this embodiment, the first to ninth wire holes (Hw11 to Hw33) have the same shape.

The bendable section 12 includes an intermediate region 12a and a bendable region 12b. The bendable region 12b is disposed at the distal end of the bendable section 12. In the bendable region 12b, a first guide ring J1, a second guide ring J2, and a third guide ring J3 are disposed. The bendable region 12b refers to a region where the degree and orientation of the bend of the bendable section 12 can be controlled by moving the first guide ring J1, the second guide ring J2, and the third guide ring J3 using the bend driving section 13. FIG. 5B is illustrated with part of the bendable section 12 covering the first to third guide rings (J1 to J3) omitted.

In this embodiment, the bendable section 12 includes multiple auxiliary rings (not shown). In the bendable region 12b, the first guide ring J1, the second guide ring J2, and the third guide ring J3 are fixed to the wall of the bendable section 12. In this embodiment, the multiple auxiliary rings are disposed at a position closer to the proximal end than the first guide ring J1, a position between the first guide ring J1 and the second guide ring J2, and at a position between the second guide ring J2 and the third guide ring J3.

The medical device is guided to the distal end of the catheter 11 by the passage Ht, the first to third guide rings (J1 to J3), and the multiple auxiliary rings.

The first to ninth drive wires (W11 to W33) are individually fixed to the first to third guide rings (J1 to J3) through the intermediate region 12a.

Specifically, the first drive wire W11, the second drive wire W12, and the third drive wire W13 are fixed to the first guide ring J1 through the multiple auxiliary rings. The fourth drive wire W21, the fifth drive wire W22, and the sixth drive wire W23 are fixed to the second guide ring J2 through the first guide ring J1 and the multiple auxiliary rings. The seventh drive wire W31, the eighth drive wire W32, and the ninth drive wire W33 are fixed to the third guide ring J3 through the first guide ring J1, the second guide ring J2, and the multiple auxiliary rings. The catheter apparatus 1 may bend the bendable section 12 in directions crossing the extending direction of the catheter 11 by driving the bend driving section 13 using the wire driving section 300. Specifically, the bendable region 12b of the bendable section 12 may be bent in directions crossing the extending direction via the first to third guide rings (J1 to J3) by moving the first to ninth drive wires (W11 to W33) in the extending direction of the bendable section 12.

The user may insert the catheter 11 into a target portion in the subject by using at least one of manually moving the catheter apparatus 1, moving the catheter apparatus 1 using the stage 2a, and bending the bendable section 12.

Although in this embodiment the bendable section 12 is bent by moving the first to third guide rings (J1 to J3) using the first to ninth drive wires (W11 to W33), this is illustrative only. One or two of the first to third guide rings (J1 to J3) and drive wires fixed thereto may be omitted.

For example, the catheter 11 may be configured without the first to sixth drive wires (W11 to W23) and the first to second guide rings (J1 to J2) and with only the seventh to ninth drive wires (W31 to W33) and the third guide ring J3. Alternatively, the catheter 11 may be configured without the first to third drive wires (W11 to W13) and the first guide ring J1 and with only the fourth to ninth drive wires (W21 to W33) and the second to third guide rings (J2 to J3).

The catheter 11 may drive one guide ring using two drive wires. Also in this case, the number of guide rings may be one or more.

### Catheter Unit

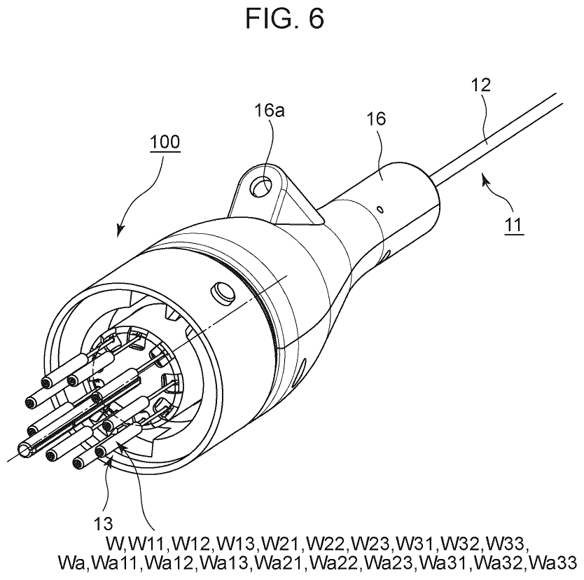

Referring to FIG. 6, the catheter unit 100 will be described. FIG. 6 is an explanatory diagram of the catheter unit 100.

The catheter unit 100 includes the catheter 11 including the bendable section 12, the bend driving section 13, the proximal end cover 16 that supports the proximal end of the catheter 11, and the first to ninth drive wires (W11 to W33) (multiple drive wires).

The proximal end cover (a frame, a bendable section casing, or a catheter casing) 16 covers part of the catheter 11. The proximal end cover 16 includes a tool hole 16a for inserting a medical device into the passage Ht of the bendable section 12.

As illustrated in FIG. 6, the first to ninth drive wires (W11 to W33) are arranged along a circle (a virtual circle) with a predetermined radius and are connected to the wire driving section 300 with the connector 21 as described below.

### Base Unit

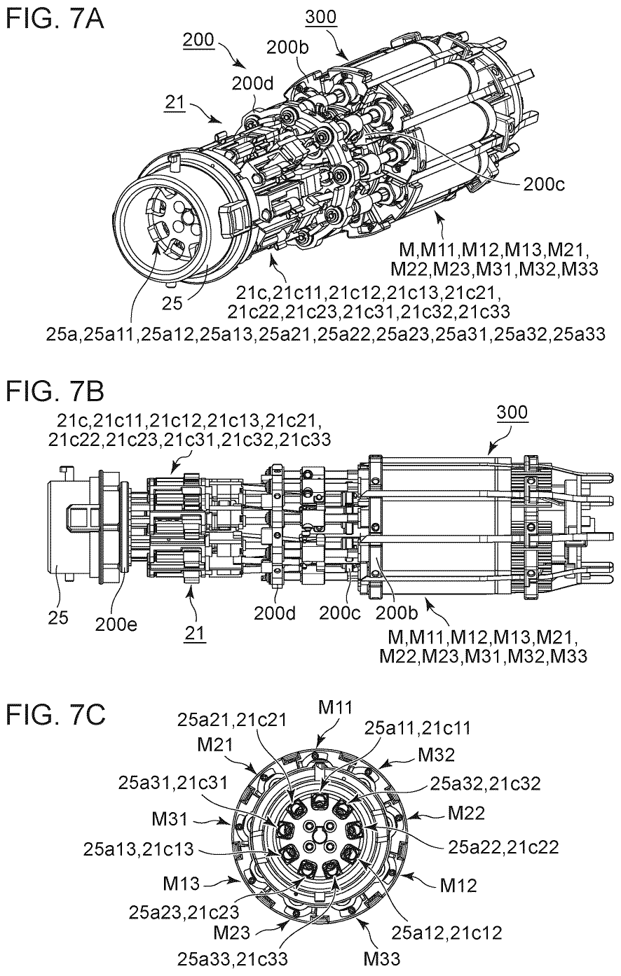

FIGS. 7A, 7B, and 7C are explanatory diagrams of the base unit 200 and the wire driving section 300. FIG. 7A is a perspective view of the base unit 200 illustrating its internal structure. FIG. 7B is a side view of the base unit 200 illustrating its internal structure. FIG. 7C is a front view of the base unit 200 seen from the catheter 11 side.

The catheter apparatus 1 includes the base unit 200 and the wire driving section 300, as described above. In this embodiment, the wire driving section 300 is housed in the base casing 200f and provided in the base unit 200. In other words, the base unit 200 includes the wire driving section 300.

The wire driving section 300 includes multiple driving sources (motors). In this embodiment, the wire driving section 300 includes a first driving source M11, a second driving source M12, a third driving source M13, a fourth driving source M21, a fifth driving source M22, a sixth driving source M23, a seventh driving source M31, an eighth driving source M32, and a ninth driving source M33.

Any one of the first to ninth driving sources (M11 to M33) may be referred to as a driving source M. In this embodiment, the first to ninth driving sources (M11 to M33) have the same configuration.

The base unit 200 includes the connector 21. The connector 21 is housed in the base casing 200f. The connector 21 is connected to the wire driving section 300. The connector 21 includes multiple connecting sections. In this embodiment, the connector 21 includes a first connecting section 21c11, a second connecting section 21c12, a third connecting section 21c13, a fourth connecting section 21c21, a fifth connecting section 21c22, a sixth connecting section 21c23, a seventh connecting section 21c31, an eighth connecting section 21c32, and a ninth connecting section 21c33.

Any one of the first to ninth connecting sections (21c11 to 21c33) may be referred to as a connecting section 21c. In this embodiment, the first to ninth connecting sections (21c11 to 21c33) have the same configuration.

Each of the multiple connecting sections is connected to a corresponding one of the multiple driving sources and is driven by driven by the corresponding one of the multiple driving sources. Specifically, the first connecting section 21c11 is connected to the first driving source M11 and is driven by the first driving source M11. The second connecting section 21c12 is connected to the second driving source M12 and is driven by the second driving source M12. The third connecting section 21c13 is connected to the third driving source M13 and is driven by the third driving source M13. The fourth connecting section 21c21 is connected to the fourth driving source M21 and is driven by the fourth driving source M21. The fifth connecting section 21c22 is connected to the fifth driving source M22 and is driven by the fifth driving source M22. The sixth connecting section 21c23 is connected to the sixth driving source M23 and is driven by the sixth driving source M23. The seventh connecting section 21c31 is connected to the seventh driving source M31 and is driven by the seventh driving source M31. The eighth connecting section 21c32 is connected to the eighth driving source M32 and is driven by the eighth driving source M32. The ninth connecting section 21c33 is connected to the ninth driving source M33 and is driven by the ninth driving source M33.

The connector 21 connects to the bend driving section 13 including the first to ninth drive wires (W11 to W33), as discussed below. The bend driving section 13 bends the bendable section 12 using a driving force of the wire driving section 300 received via the connector 21.

The drive wires W are connected to the connecting sections 21c via the retained portions Wa. Each of the multiple drive wires W is connected to a corresponding one of the multiple connecting sections 21c.

Specifically, the first retained portion Wa11 of the first drive wire W11 is connected to the first connecting section 21c11. The second retained portion Wa12 of the second drive wire W12 is connected to the second connecting section 21c12. The third retained portion Wa13 of the third drive wire W13 is connected to the third connecting section 21c13. The fourth retained portion Wa21 of the fourth drive wire W21 is connected to the fourth connecting section 21c21. The fifth retained portion Wa22 of the fifth drive wire W22 is connected to the fifth connecting section 21c22. The sixth retained portion Wa23 of the sixth drive wire W23 is connected to the sixth connecting section 21c23. The seventh retained portion Wa31 of the seventh drive wire W31 is connected to the seventh connecting section 21c31. The eighth retained portion Wa32 of the eighth drive wire W32 is connected to the eighth connecting section 21c32. The ninth retained portion Wa33 of the ninth drive wire W33 is connected to the ninth connecting section 21c33.

The base unit 200 includes a base frame 25. The base frame 25 has multiple insertion holes for passing the first to ninth drive wires (W11 to W33) therethrough individually. The base frame 25 includes a first insertion hole 25a11, a second insertion hole 25a12, a third insertion hole 25a13, a fourth insertion hole 25a21, a fifth insertion hole 25a22, a sixth insertion hole 25a23, a seventh insertion hole 25a31, an eighth insertion hole 25a32, and a ninth insertion hole 25a33. The first to ninth insertion holes (25a11 to 25a33) correspond to the first to ninth drive wires (W11 to W33), respectively. The numerals following the sign 25a represent the corresponding drive wires. For example, the first drive wire W11 is inserted into the first insertion hole 25al1.

Any one of the first to ninth insertion holes (25a11 to 25a33) may be referred to as an insertion hole 25a. In this embodiment, the first to ninth insertion holes (25a11 to 25a33) have the same shape.

The base unit 200 further includes a motor frame 200b, a first bearing frame 200c, a second bearing frame 200d, and a third bearing frame 200e. The motor frame 200b, the first bearing frame 200c, the second bearing frame 200d, and the third bearing frame 200e are joined together. Connecting Sections between Motor and Drive Wire

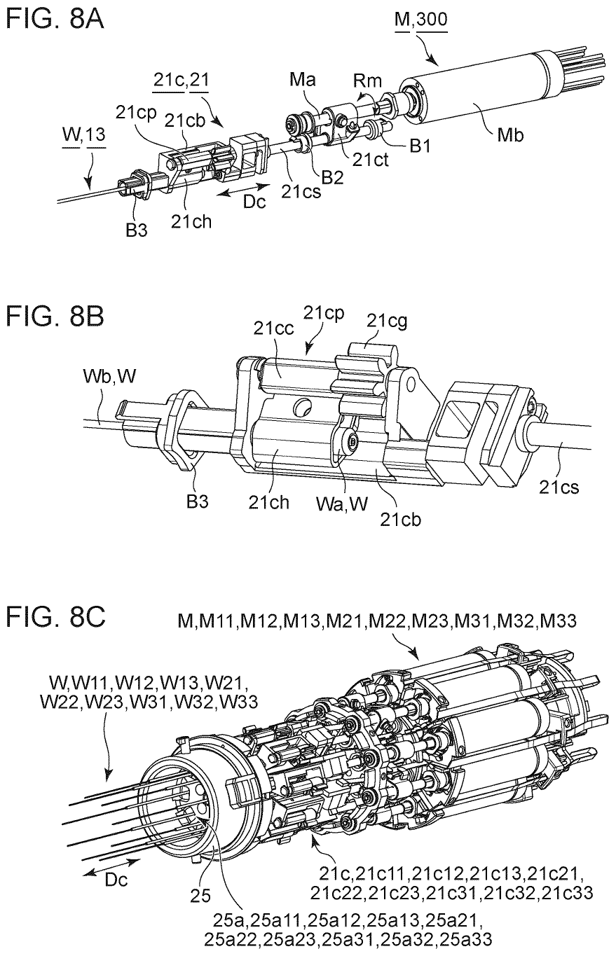

Referring to FIGS. 8A, 8B, and 8C, the connection of the wire driving section 300, the connector 21, and the bend driving section 13 will be described. FIGS. 8A, 8B, and 8C are explanatory diagrams of the wire driving section 300, the connector 21, and the bend driving section 13. FIG. 8A is a perspective view of the driving source M, the connecting section 21c, and the drive wire W. FIG. 8B is an enlarged view of the connecting section 21c and the drive wire W. FIG. 8C is a perspective view of the connection of the wire driving section 300, the connector 21, and the bend driving section 13.

In this embodiment, the configurations of the connections between the first to ninth drive wires (W11 to W33) and the corresponding first to ninth connecting sections (21c11 to 21c33) are the same. The configurations of the connections between the first to ninth connecting sections (21c11 to 21c33) and the corresponding first to ninth driving sources (M11 to M33) are the same. Accordingly, the configuration of the connection of a single drive wire W, a single connecting section 21c, and a single driving source M will be described hereinbelow.

As illustrated in FIG. 8A, the driving source M includes an output shaft Ma (a motor shaft) and a motor main body Mb that rotates the output shaft Ma in a rotational direction Rm. The output shaft Ma has a helical groove in its surface. The output shaft Ma has a what-is-called screw shape. The motor main body Mb is fixed to the motor frame 200b.

The connecting section 21c includes a tractor 21ct connected to the output shaft Ma and a tractor support shaft 21cs that supports the tractor 21ct. The tractor support shaft 21cs is connected to a connection base 21cb.

The connecting section 21c includes a leaf spring 21ch serving as a retainer for retaining the retained portion Wa of the drive wire W. The drive wire W passes through the insertion hole 25a and engages with the connecting section 21c. More specifically, the retained portion Wa engages with the leaf spring 21ch. The leaf spring 21ch holds and fixes the retained portion Wa as discussed below.

The connecting section 21c includes a pressing member 21cp. The pressing member 21cp includes a gear 21cg to be engaged with an internal gear 29 (not shown) and a cam 21cc serving as a pressing section for pressing the leaf spring 21ch.

The cam 21cc is movable relative to the leaf spring 21ch. The cam 21cc is moved as the internal gear 29 (not shown) rotates to fix the leaf spring 21ch.

The connecting section 21c is supported by a first bearing B1, a second bearing B2, and a third bearing B3. The first bearing B1 is supported by the first bearing frame 200c of the base unit 200. The second bearing B2 is supported by the second bearing frame 200d of the base unit 200. The third bearing B3 is supported by the third bearing frame 200e of the base unit 200. This prevents the connecting section 21c from rotating around the output shaft Ma when the output shaft Ma rotates in the rotational direction Rm. The first bearing B1, the second bearing B2, and the third bearing B3 are provided for each of the first to ninth connecting sections (21c11 to 21c33).

Since the rotation of the connecting section 21c around the output shaft Ma is restricted, the rotation of the output shaft Ma causes a force along the axis of rotation of the output shaft Ma to act on the tractor 21ct due to the helical groove of the output shaft Ma. As a result, the connecting section 21c moves along the axis of rotation of the output shaft Ma (in the direction of Dc). The movement of the connecting section 21c causes the drive wire W to move, thereby bending the bendable section 12.

In other words, the output shaft Ma and the tractor 21ct constitute a feed screw that converts the rotary motion transmitted from the driving source M to linear motion using the screw. The output shaft Ma and the tractor 21ct in this embodiment constitute a sliding screw but may be a ball screw.

As illustrated in FIG. 8C, by mounting the catheter unit 100 on the base unit 200, the first to ninth drive wires (W11 to W33) are connected to the first to ninth connecting section (21c11 to 21c33), respectively.

The controller 3 controls the first to ninth driving sources (M11 to M33) independently. In other words, any one of the first to ninth driving sources (M11 to M33) may be operated or stopped independently regardless of whether the other driving sources are at rest. In other words, the controller 3 may control the first to ninth drive wires (W11 to W33) independently. As a result, the first to third guide rings (J1 to J3) are controlled independently to bend the bendable region 12b of the bendable section 12 in any direction.

### Catheter Apparatus and Movable Range of Catheter

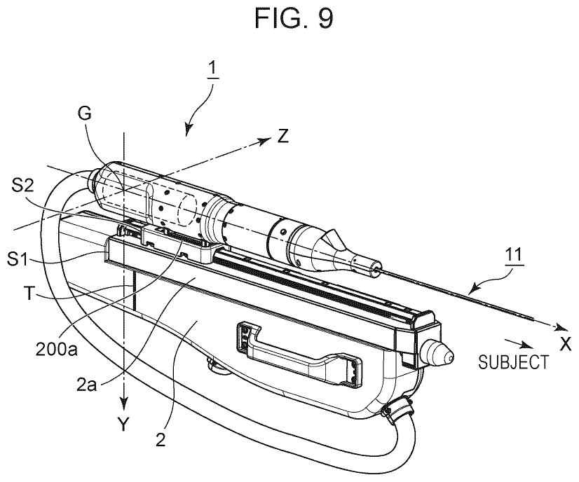

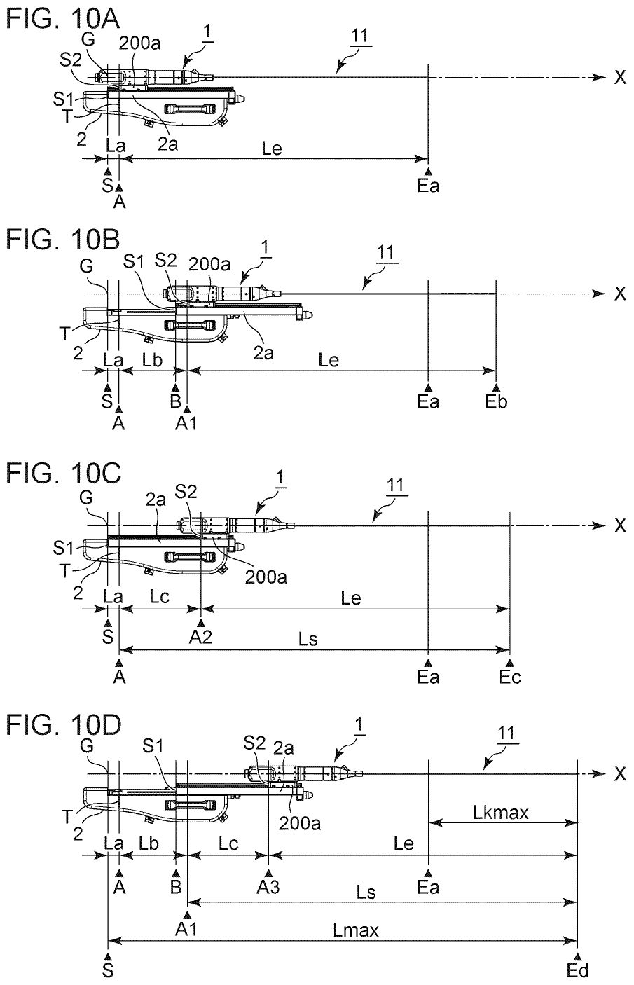

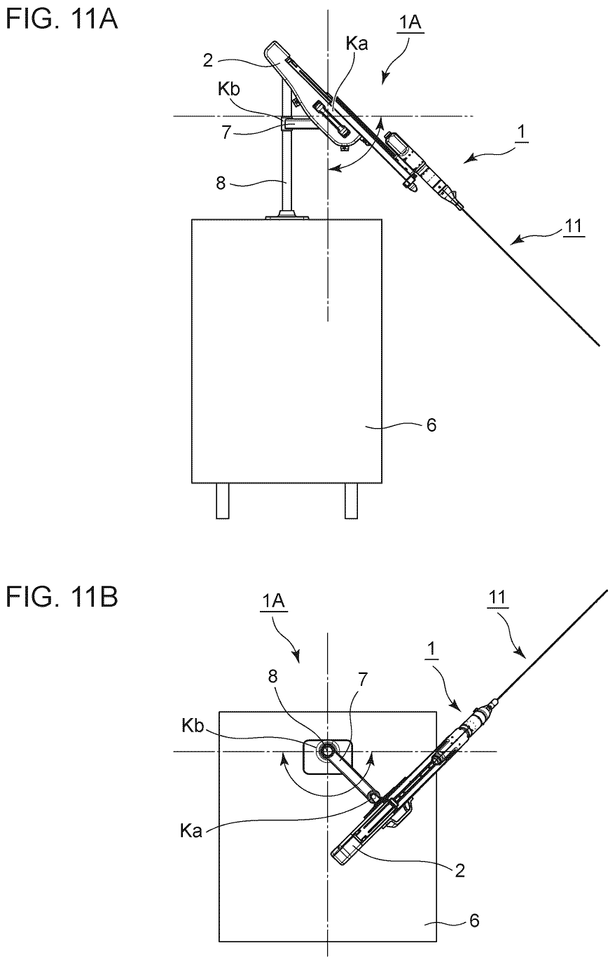

Referring to FIG. 9, FIGS. 10A to 10D, and FIGS. 11A and 11B, the operating ranges of the catheter apparatus 1 and the catheter 11 will be described. FIG. 9 is a perspective view of the catheter apparatus 1 illustrating coordinate setting in the moving direction of the catheter apparatus 1 and the stage 2a in the following description. The X-axis extends in the direction in which the catheter apparatus 1 moves straight ahead on the support base 2. The X-axis passes through the central axis of the catheter 11. The direction in which the catheter 11 is inserted into the subject is referred to as the positive direction of the X-axis. The axis perpendicular to the X-axis is the Y-axis. The Y-axis extends from the X-axis in the direction perpendicular to the ground (in the vertical direction) when the X-axis is parallel to the ground (the installation surface of the medical system 1A) (when the (X-axis is in the horizontal direction). The stage 2a has an abutment surface S1 that comes into contact with the support base 2 (referred to as a back surface of the stage 2a) at the position to which the stage 2a has moved maximum (the furthest position) in the negative direction of the X-axis. The Y-axis passes through the abutment surface S1 (is aligned with the abutment surface S1) at the furthest position in the negative direction of the X-axis. The direction from the catheter apparatus 1 to the ground when the X-axis is parallel to the ground is referred to as the positive direction of the Y-axis. The intersection of the X-axis and the Y-axis is referred to as an origin G. The Z-axis passes through the origin G and extends in the direction perpendicular to the plane formed by the X-axis and the Y-axis. The direction from the lower left to the upper right in FIG. 9 is defined as the positive direction of the Z-axis.

The support base 2 displays information for the user to recognize the origin G and a position equivalent to the origin G. In this embodiment, the support base 2 includes a position display section that displays information for the user to recognize that the catheter apparatus 1 (the attaching section 200a) is at the furthest position in the negative direction of the X-axis. In this embodiment, when the catheter apparatus 1 (attaching section 200a) is at the furthest position in the negative direction of the X-axis, the abutment surface S2 (referred to as the back surface of the attaching section 200a) comes into contact with the stage 2a to reach a position A. A linear marking T, serving as the position display section, is disposed at position A on the side of the case of the support base 2. The user can recognize that the catheter apparatus 1 (the attaching section 200a) is at the furthest position in the negative direction of the X-axis when the back surface S2 aligns with the linear marking T. The position display section is not limited to the linear marking T. For example, a light emitting diode (LED) may be provided on the side of the case of the support base 2 to allow the user to recognize that the catheter apparatus 1 (the attaching section 200a) is at the furthest position in the negative direction of the X-axis using light.

FIGS. 10A to 10D are explanatory diagrams of the linear motion range related to the catheter apparatus 1 on the support base 2. FIG. 10A is a side view of the catheter apparatus 1 at the initial position. FIG. 10B is a side view of the stage 2a illustrating its movable range. FIG. 10C is a side view of the catheter apparatus 1 illustrating the range in which the attaching section 200a is attachable onto the stage 2a. FIG. 10D is a side view of the catheter apparatus 1 in the most extending state.

In FIG. 10A, the catheter apparatus 1 is at the initial position. At the initial position, the stage 2a is at the furthest position in the negative direction of the X-axis relative to the support base 2 within the movable range on the support base 2 (at the retracted position of the stage 2a or a first retracted position). At this time, the back surface S1 of the stage 2a is at position S. The position S is at the same location as the origin G in the X-axis direction.

The catheter apparatus 1 may be mounted on multiple locations in a predetermined region of the stage 2a via the attaching section 200a. The catheter apparatus 1 (the attaching section 200a) is movable relative to the stage 2a in the predetermined region of the stage 2a. The predetermined region may also be referred to as the movable range of the catheter apparatus 1 or the attachable range of the attaching section 200a. At the initial position, the catheter apparatus 1 (the attaching section 200a) is at the furthest position in the negative direction of the X-axis relative to the stage 2a (at the retracted position of the catheter apparatus 1 or the attaching section 200a, a second retracted position, or a trailing end position). The back surface S2 of the attaching section 200a is at position A. The difference between the back surface S1 of the stage 2a and the back surface S2 of the attaching section 200a (the distance between position S and position A) is defined as an initial positional difference La. The position S is at the same location as the origin G in the X-axis direction. Accordingly, the distance between position A and the marking T located at position A and the origin G is equal to the initial positional difference La. In the state of FIG. 10A, the distal end of the catheter 11 is at the furthest position in the negative direction of the X-axis within the movable range of the catheter apparatus 1 in the X-axis direction. The distal end of the catheter 11 at that time is at position Ea. The length from the back surface S2, which is part of the attaching section 200a), to the distal end of the catheter 11 (the length between position A and position Ea) is referred to as Le. The back surface S2 of the attaching section 200a serves as a reference for the length to the distal end of the catheter 11 in the catheter apparatus 1. The length Le is fixed regardless of the position of the stage 2a relative to the support base 2 and the position of the catheter apparatus 1 relative to the stage 2a.

As illustrated in FIG. 10B, the stage 2a is at the most forward position in the positive direction of the X-axis relative to the support base 2 within the movable range on the support base 2 (the forward position of the stage 2a or a first forward position). The stage 2a is movable relative to the support base 2 between the first forward position and the first retracted position. The distance between the distal end of the catheter 11 and the support base 2 is longer in the extending direction of the catheter 11 when the stage 2a is at the first forward position than when at the first retracted position. The back surface S1 of the stage 2a is at position B. In other words, the stage 2a is movable to any position between the position where the back surface S1 is at position S and the position where the back surface S1 is at position B. The distance between position S and position B is referred to as a maximum linear travel distance Lb. At this time, in FIG. 10B, the back surface S2 of the attaching section 200a is at position A1. The distal end of the catheter 11 at this time is at position Eb. The length Le is fixed regardless of the position of the stage 2a. The distance to the distal end of the catheter 11 from the origin G (the distance between position S and position Eb) may be given by Lb+La+Le.

Referring to FIG. 10C, the catheter apparatus 1 (the attaching section 200a) is at the furthest position in the positive direction of the X-axis relative to the stage 2a within the movable range on the stage 2a (at the forward position of the catheter apparatus 1 or the attaching section 200a, a second forward position, or a leading end position). At this time, the back surface S2 of the attaching section 200a is at position A2. The attaching section 200a is attachable onto the stage 2a between the position where the back surface S2 is at position A and the position where the back surface S2 is at position A2, with the stage 2a located at the retracted position (the back surface S2 of the stage 2a located at position S). In other words, the catheter apparatus 1 (the attaching section 200a) is attachable onto the stage 2a between the second forward position and the second retracted position. The second forward position and the second retracted position themselves are also included in the attaching region. The distance between the distal end of the catheter 11 and the support base 2 is longer in the extending direction of the catheter 11 when the catheter apparatus 1 (the attaching section 200a) is at the second forward position than when at the second retracted position. The distance between position A and position A2 is referred to as a maximum attaching distance Lc. The maximum attaching distance Lc is the same as the length of the movable range and may also be referred to as the distance between the second retracted position and the second forward position. In other words, the maximum attaching distance Lc is the length of the region (attaching region) in which the catheter apparatus 11 (the attaching section 200a) is attachable to the stage 2a. In FIG. 10C, the distal end of the catheter 11 is at position Ec. The length Le is fixed regardless of the position of the attaching section 200a. The distance to the distal end of the catheter 11 from the origin G (the distance between position S and position Ec) may be given by Lc+La+Le.

In FIG. 10D, the catheter apparatus 1 is at the most extending position (hereinafter referred to as the furthest position of the catheter apparatus 1). In other words, FIG. 10D illustrates the catheter apparatus 1 at the furthest position in the positive direction of the X-axis relative to the support base 2. At this time, the back surface S1 of the stage 2a is at position B, and the back surface S2 of the attaching section 200a is at position A3. At this time, the distal end of the catheter 11 is at position Ed. The length Le is fixed regardless of the positions of stage 2a and the attaching section 200a. The distance to the distal end of the catheter 11 from the origin G (the distance between position S and position Ed) may be given by La+Lb+Lc+Le. At this time, the distance to the distal end of the catheter 11 from the origin G is referred to as the maximum reach distance Lmax of the catheter apparatus 1.

As illustrated in FIGS. 10A and 10D, the catheter apparatus 1 is movable between the maximum retracted position (the retracted position of the catheter apparatus 1) and the maximum forward position (the forward position of the catheter apparatus 1) relative to the support base 2. In this embodiment, in the state in which the catheter apparatus 1 is at the maximum retracted position relative to the support base 2, the stage 2a is at the first retracted position relative to the support base 2 and the catheter apparatus 1 (the attaching section 200a) is at the second retracted position relative to the stage 2a. At this time, the distal end (leading end) of the catheter 11 is closest to the support base 2. In this embodiment, in the state in which the catheter apparatus 1 is at the maximum forward position relative to the support base 2, the stage 2a is at the first forward position relative to the support base 2, and the catheter apparatus 1 (the attaching section 200a) is at the second forward position relative to the stage 2a. At this time, the distal end of the catheter 11 is furthest from the support base 2. In other words, the distance between distal end of the catheter 11 and the support base 2 in the extending direction of the catheter 11 is longer when the catheter apparatus 1 is at the maximum forward position relative to the support base 2 than when the catheter apparatus 1 is at the maximum retracted position relative to the support base 2.

The distance the distal end of the catheter 11 moves (the distance between position Ea and position Ed) is given by Lb+Lc. This distance is referred to as the maximum displacement Lkmax of the distal end of the catheter 11.

The maximum reach distance Lmax of the catheter apparatus 1 and the maximum displacement Lkmax of the distal end of the catheter 11 may be fixed values depending on the apparatus configuration. In this embodiment, information on the maximum reach distance Lmax of the catheter apparatus 1 and the maximum displacement Lkmax of the distal end of the catheter 11 are stored in advance in the storage device 2703 of the controller 3. The controller 3 obtains the information on the maximum linear travel distances of the catheter apparatus 1 and the catheter 11 by reading the stored data. The information on the maximum reach distance Lmax and the maximum displacement Lkmax may be given using various input methods. For example, the user may manually input the values written in a user manual via an input device such as a touch screen of the monitor 4 or a keyboard. Other examples include a method of scanning a barcode displayed on the catheter apparatus 1, a method of downloading the information via a network, and a method for obtaining the information from a medium such as a universal serial bus (USB) memory.

FIGS. 11A and 11B illustrate the relationship of the movable range among the support base 2, the arm 7, and the catheter 11. FIG. 11A is a side view of the medical system 1A illustrating the relationship of the movable range between the support base 2 and the catheter apparatus 1. FIG. 11B is a top view of the medical system 1A illustrating the relationship of the movable range between the arm 7 and the catheter apparatus 1.

As illustrated in FIG. 11A, the support base 2 is connected to the arm 7 so as to be rotatable around a joint Ka serving both as a connection to the arm 7 and the rotation axis of the support base 2. The direction of rotation of the support base 2 around the joint Ka relative to the arm 7 is a direction in which the X-axis is inclined downward in the positive direction of the X-axis from the direction parallel to the installation surface of the medical system 1A (the X-axis is horizontal), with the support base 2 supporting the lower part of the catheter apparatus 1. In this embodiment, the support base 2 is rotatable around the joint Ka relative to the arm 7 within a rotation angle of 90 degrees. Accordingly, the support base 2 is rotatable to the position where the positive direction of the X-axis is vertically downward.

As illustrated in FIG. 11B, the arm 7 serves as a connection to the support shaft 8 and is connected to the support shaft 8 so as to be rotatable around a joint Kb serving as the rotation axis of the arm 7. The direction of rotation of the arm 7 around the joint Kb around the support shaft 8 is parallel to the installation surface of the medical system 1A, and the arm 7 is rotatable to the right and left within a rotation angle of 90 degrees (the maximum rotation angle is 180 degrees, where the position at which the longitudinal direction of the arm 7 is perpendicular to the front of the medical system 1A is defined as 0 degree).

Accordingly the three-dimensional movable range of the distal end of the catheter 11 may be represented as a quadrant of a sphere having a hollow center in which the movable ranges of the catheter apparatus 1, the support base 2, and the arm 7 illustrated in FIGS. 10A to 10D, and FIGS. 11A and 11B are totaled. Among them, the locus of the distal end of the catheter 11 when the catheter apparatus 1 is at the furthest position is the three-dimensional maximum movable range of the catheter 11.

Thus, the user may move and locate the catheter apparatus 1 to any desired position within the respective movable ranges of the attaching section 200, the stage 2a, the support base 2, and the arm 7.

### Estimating Lumen Path Length in Patient's Body

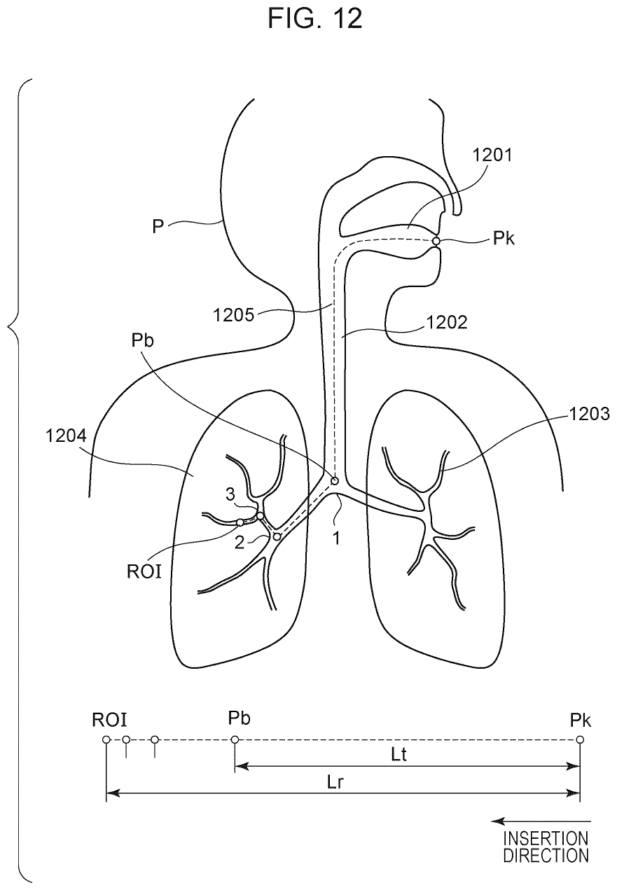

FIG. 12 is a diagram illustrating an example of medical image data. A method for estimating a lumen path length Lr in the patient's body will be described hereinbelow with reference to FIG. 12. FIG. 12 is an example of a display screen that displays a three-dimensional (3D) model in the patient1 body and navigation of a path for inserting the catheter 11 to the ROI in the patient's body illustrated in the 3D model.

As illustrated in FIG. 12, the controller 3 generates a 3D model from preoperative chest computed tomography (CT) scan data of the patient. In this embodiment, the display range of the 3D model is the whole of the lungs from the patient's mouth Pk serving as the approach point (insertion start point) of the catheter 11 through the oral cavity 1201 and the trachea 1202 to the deep part of the bronchial tube 1203. The approach point of the catheter 11 may be any start point of the path through which the catheter 11 can enter the trachea and may be not only the patient's mouth but also nose. The generation range of the 3D model may be restricted to a partial area in the body. For example, in the case of a 3D model of only part of the lungs 1204 starting from the point where the tracheal branches off into the bronchial tube, information on the path length 1205 from the patient's mouth Pk through the oral cavity 1201 to the bronchial tube 1203 is insufficient. For path estimation using a 3D model with a restricted region, the insufficient regional information on the body lumen is interpolated by supplementary information. The supplementary information may be values (data) obtained by measuring the patient1 body before the surgery or values estimated from data, etc. based on the information unique to the patient, such as the body height. The method by which the controller 3 obtains the interpolation information is not limited to manual input by the user through an input device such as touch input on the monitor 4 or a keyboard. For example, the controller 3 may obtain the interpolation information by scanning a unique bar code written on the patient's record, via a network, or from a medium such as a USB memory.

In this embodiment, the CPU 2701 of the controller 3 plans a stereoscopic path from the patient's mouth Pk to the ROI in the generated 3D model and calculates a lumen path length Lr in the patient's body necessary for the distal end of the catheter 11 to reach the ROI. Specifically, as in the example illustrated in FIG. 12, the CPU 2701 determines a path from the patient's mouth Pk through the oral cavity, the trachea, and branches 1 to 3 in the bronchial tube to the ROI and calculates the lumen path length Lr in the patient1 body. In FIG. 12, the first branch 1 is represented as a branch point Pb. The CPU 2701 calculates the path distance Lt between the mouth Pk and the branch point Pb and stores the path distance Lt in the memory 2702 or the storage device 2703. The CPU 2701 also stores the lumen path length Lr described before in the memory 2702 or the storage device 2703. The CPU 2701 reads the lumen path length Lr and the path distance Lt stored in the memory 2702 or the storage device 2703 as needed for display processing on the monitor 4. Since the position of the ROI in the lumen path in the patient1 body varies depending on the patient, the 3D model or the planed path may be independently set.

In this embodiment, generation of the 3D model, planning of the approach path of the catheter 11, and calculation of the path lengths are performed by the controller 3. The controller 3 may obtain part or all of data generated outside the medical system 1A such as other applications. If the controller 3 obtains only some of data from the outside, the controller 3 may address the shortage by calculating and interpolating the deficient data.

Although this embodiment illustrates the planning method using a 3D model related to the patient's chest lumen, a 3D model and a planning method for the lumen path length Lr similar to those of this embodiment are applicable also to a patient's lumen other than the chest lumen.

### Installation Navigation for Catheter Apparatus

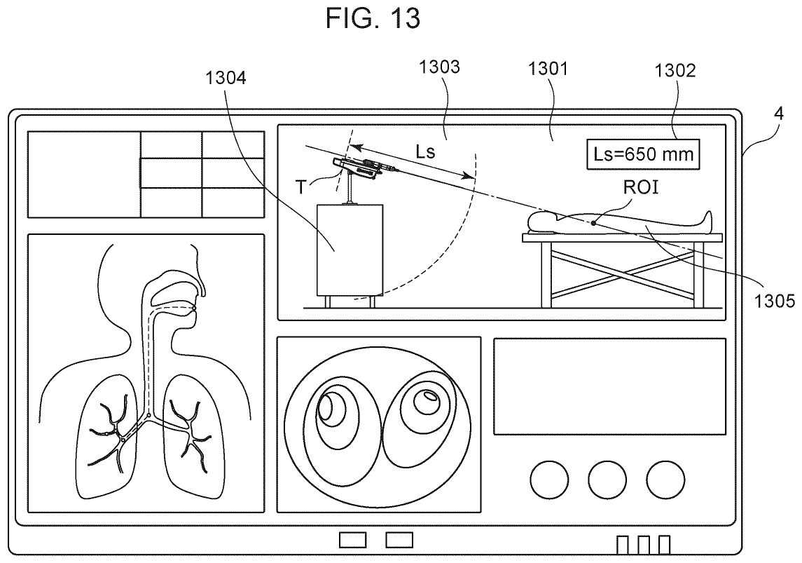

Installation navigation for the catheter apparatus 1 will be described with reference to FIG. 13. FIG. 13 illustrates an example of the display screen of the monitor 4 that displays installation navigation for the catheter apparatus 1.

When adjusting the positioning of the catheter apparatus 1 and the medical system 1A, the user adjusts the position of the catheter apparatus 1 while visually checking the catheter apparatus 1 and the patient's mouth. In this embodiment, information related to the distal end position of the catheter 11 (reach information) is reported using the monitor 4 (notification section). The reach information is information related to the position of the distal end (leading end) of the catheter 11.

As illustrated in FIG. 13, reach length information 1302 and Ls visual information 1303 are displayed in a notification area 1301. The Ls visual information 1303 includes an illustration that shows the reachable range of the distal end of the catheter 11. Both of the reach length information 1302 and the Ls visual information 1303 may be referred to as reach information related to the position of the distal end (leading end) of the catheter 11. In this embodiment, as illustrated in FIG. 13, a reachable distance Ls is displayed together with an illustration of the catheter apparatus 1 attached to the second forward position (the position where the back surface S2 of the attaching section 200a aligns with position A2) relative to the stage 2a. In contrast, the stage 2a is at the first retracted position relative to the support base 2. In other words, in this embodiment, the reach length information 1302 and the Ls visual information 1303 include information related to the position of the distal end of the catheter 11 when the catheter apparatus 1 is at the second forward position relative to the stage 2a, and the stage 2a is at the first retracted position relative to the support base 2. The Ls visual information 1303 also displays the relationship among the reachable range of the distal end of the catheter 11 (for example, the reachable distance Ls), a medical system 1304 (1A), the marking T, and a patient 1305 who is the subject of the catheter 11. More specifically, the Ls visual information 1303 displays the reachable distance Ls as the information related to the length from the marking T, which is part of the support base 2, to the distal end of the catheter 11. The reachable distance Ls is the length of the catheter 11 in the extending direction. In this embodiment, the reachable distance Ls is equal to the sum of Lc and Le. Displaying the relationship between the reachable distance Ls and the marking T allows the user to easily determine which part of the support base 2 the reachable distance Ls is referenced from. Displaying the relationship between the reachable distance Ls and the patient 1305 allows the user to determine how to dispose the medical system 1A relative to the patient 1305. In addition to or in place of the reachable distance Ls, information related to the position of the distal end of the catheter 11 when the catheter apparatus 1 is at the maximum forward position relative to the support base 2 may be displayed. In this way, by looking at the notification area 1301, the user can easily determine how close to dispose the medical system 1A and the catheter apparatus 1 relative to the patient 1305. This allows for supporting the user in disposing the medical system 1A and the catheter apparatus 1 relative to the patient.

The reach information may be the individual Lc and Le or the reachable distance Ls that is the sum of Lc and Le. Thus, the user may check the reach information displayed on the monitor 4 and adjust the length from the marking T on the support base 2 to the bronchial tube through the oral cavity or the nasal cavity of the patient to fall within the reachable distance Ls by moving the arm 2 and cart 6.

The reachable distance Ls displayed on the monitor 4 does not have to be equal to (Lc+Le) and may be approximately (Lc+Le).

For example, the reachable distance Ls may be equal to or less than (Lc+Le). Displaying a length equal to or less than (Lc+Le) as a reference on the monitor 4 may prompt the user to move the cart 6 or the arm 2 so that the distal end of the catheter 11 may reach the initial target position (for example, the branch point Pb) more reliably. Too short reachable distance Ls is undesirable. For this reason, the reachable distance Ls is preferably 70% or more and 100% or less of (Lc+Le).

In another example, if the user uses the distance to the patient's mouth as a reference for positioning the catheter apparatus 1, a region (value) obtained by subtracting the path distance Lt between the mouth Pk and the branch point Pb from the reachable distance Ls may be displayed on the monitor 4. The user or the like performs planning for each patient and inputs the value Lt to the controller 3 via the input device 2704. The CPU 2701 calculate a reference value (Ls-Lt) to be displayed on the monitor 4 using the input value Lt and the stored reachable distance Ls. Displaying the length (Ls-Lt) or a length of 70% or more and 100% or less of (Ls-Lt) on the monitor 4 allows the user to determine whether the distal end of the catheter 11 reaches the patient's mouth, providing a more intuitive and easy-to understand support system. The user may properly locate the catheter apparatus 1 of the medical system 1A relative to the subject, for example, by moving the cart 6.

The monitor 4 may display a message to prompt the user to correct (change) the position of the medical system 1A and the catheter apparatus 1 relative to the patient. Specifically, a message describing the positioning image of the catheter apparatus 1 relative to the patient (for example, a message to place the medical system 1A and the patient within a specified range) may be additionally displayed.

### Second Embodiment

Next, a second embodiment will be described. In the first embodiment, the user is visually notified of the region (distance) that the distal end of the catheter 11 (bendable member) reaches. The second embodiment describes another method whereby the user determines the reachable range or position of the distal end of the catheter 11 from the support base 2. The same components as in the first embodiment are denoted by the same reference signs, and detailed descriptions will be omitted.

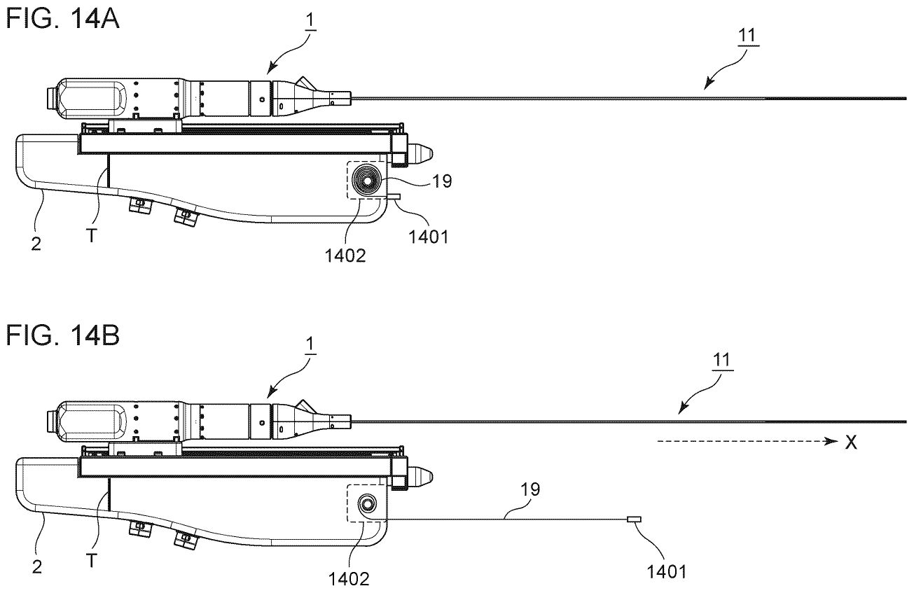

FIGS. 14A and 14B are side views of the support base 2 including a linear member 19 and the catheter apparatus 1 attached to the support base 2. As will be described below, the linear member 19 functions as a measuring device for measuring the distance to the patient (the subject into which the catheter 11 is to be inserted). The linear member 19 may also function as a verification device for confirming the distal end of the catheter 11. FIG. 14A illustrates a state in which the linear member 19 is housed in a housing 1402 of the support base 2. FIG. 14B illustrates a state in which the linear member 19 is drawn out by the user using a grip 1401. As illustrated in FIG. 14A, the linear member 19 is housed in the support base 2 in a wound form when not in use. As illustrated in FIG. 14B, the user may hold the grip 1401 of the linear member 19 to draw out the linear member 19 in the positive direction of the X-axis. Providing the medical system 1A with a fixing means for fixing the amount of draw of the linear member 19 may improve the usability.

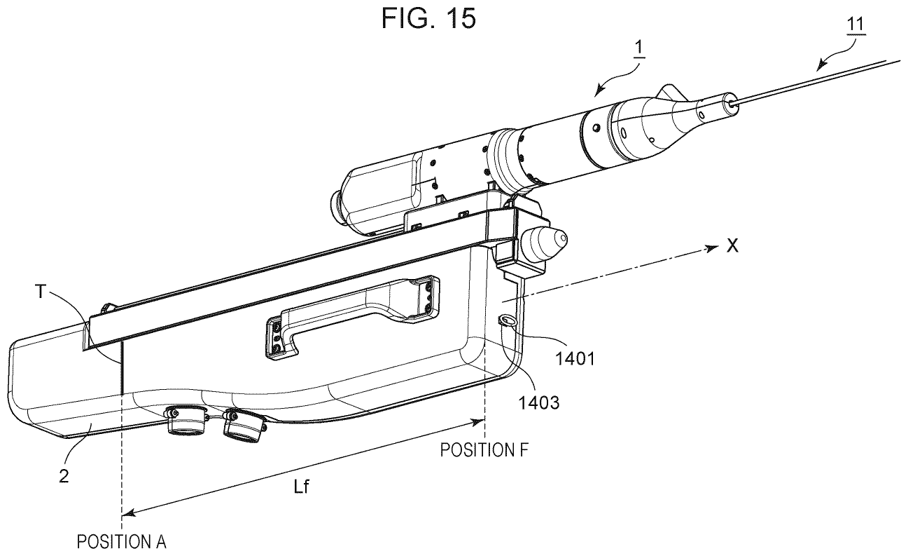

FIG. 15 is a perspective view of the support base 2 including the linear member 19 and the catheter apparatus 1 attached to the support base 2. The linear member 19 is provided on the front surface of the support base 2 in the positive direction of the X-axis. The grip 1401 is exposed from an opening 1403. The user may confirm the reachable position of the catheter 11 by holding the grip 1401 of the linear member 19 and drawing out the linear member 19 toward the patient. As illustrated in FIG. 15, the length from the linear marking T (position A) to the support base front surface F is defined as Lf. In other words, the length Lf is the length from the linear marking T to the placement position of the linear member 19. The value Lf is stored in advance in a storage so that the controller 3 can refer thereto. The value Lf may be obtained by the user manually inputting the value Lf written in a user manual via an input device such as a touch screen of the monitor 4 or a keyboard. Other examples include a method of downloading the value Lf via a network, and a method for obtaining the value Lf from a medium such as a USB memory.

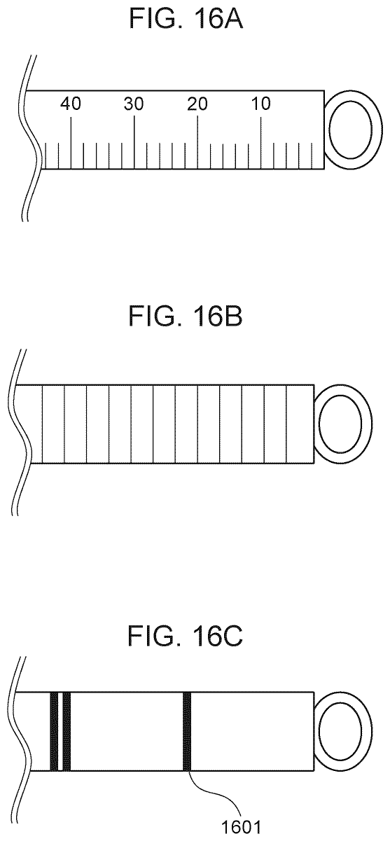

FIGS. 16A to 16C illustrate the details of the linear member 19. FIG. 16A illustrates a case where the linear member 19 is a measure (measuring means). The linear member 19 is marked with a scale indicating the length from the distal end of the catheter 11. The user may measure the distance from the support base 2 to the subject (patient's mouth Pk) by holding the grip 1401 of the linear member 19 and drawing out the linear member 19, for example, toward the patient's mouth Pk. Specifically, the user may find the distance to the patient's mouth Pk by reading the scale at the base of the linear member 19 when the distal end of the linear member 19 reaches the patient's mouth Pk. The magnitude of the scale printed on the linear member 19 relative to the actual length of the drawn linear member 19 in consideration of the difference in position between the linear member 19 and the catheter 11. The user can determine whether the positioning of the medical system 1A needs to be modified by comparing the information on the position of the distal end of the catheter 11 displayed on the monitor 4 and the result of measurement. For example, the user can determine whether the positioning of the medical system 1A needs to be modified by comparing the reachable distance Ls and the distance to the patient's mouth Pk measured using the linear member 19.

At this time, the monitor 4 displays (Ls−Lf) as the reference length for measurement in the notification area 1301. In other words, the maximum permissible length for measurement is the distance obtained by subtracting the length Lf from the reachable distance Ls (the sum of Lc and Le). In other words, the monitor 4 displays the reference length as information related to the target distance estimated using the linear member 19 serving as a measuring device. The user may determine whether the positioning of the medical system 1A needs to be modified by comparing the value (Ls−Lf) and the distance to the patient's mouth Pk measured using the linear member 19.

A reference length (Le−Lf) may be displayed for the case where the catheter apparatus 1 is attached at the second retracted position relative to the stage 2a. For example, in the case where the linear member 19 is used when the catheter apparatus 1 is not mounted on the stage 2a, the value (Le−Lf) may be used as a reference for safety. This prevents more reliably the distal end of the catheter 11 from failing to reach the desired position when the catheter apparatus 1 is attached.

The length obtained by the user pulling the grip of the linear member 19, used as a measure, to the entrance of the patient's oral cavity or nasal cavity and reading the scale is defined as Lm. Let It be the length from Pk to Pb, where Pk is the patient's mouth, and Pb is the branch point of the patient. The value (Ls−Lf−Lt) may be displayed on the monitor 4 as a reference length. If the scale on the measure begins from 0, and the measured length Lm is within (Ls−Lf−Lt), the user may determine that the distal end of the catheter 11 reaches the ROI. If (Ls−Lf−Lt)<Lm, the positioning of the apparatus and the patient is adjusted.

The scale of the measure may begin from Lf. In this case, if a length Lm′ read from the scale is within (Ls−Lt), it can be determined that the distal end of the catheter 11 may reach the ROI. If (Ls−Lt)<Lm′, the positioning of the apparatus and the patient is adjusted.

An encoder or a sensor equivalent thereto may be placed in the support frame in the vicinity of the opening 1403 to detect the amount of draw of the linear member 19, and the controller 3 may be operated based on the detection result. This encoder or an equivalent sensor corresponds to a draw amount detector that detects the amount of draw of the linear member 19.

For example, the linear member 19 is marked with a scale as illustrated in FIG. 16B. The draw-amount detection sensor optically reads the passing scale as the linear member 19 is drawn out. More specifically, the optical sensor detects the passing scale when the linear member 19 is drawn out from the opening 1403 or returned to the housing 1402 by the user. The magnitude of the scale printed on the linear member 19 relative to the actual draw length of the linear member 19 may be adjusted in consideration of the difference in position between the linear member 19 and the catheter 11. The detected signal is input to the controller 3 via the input device 2704. The CPU 2701 counts the scale, converts the amount of draw to a length according to the count, and displays the converted length on the monitor 4 (notification section) as visualized information. In other words, the monitor 4 displays information related to the distance to the subject measured by the linear member 19 serving as a measuring device. The CPU 2701 determines whether the converted draw length is within a predetermined value such as (Ls−Lf), and if the converted draw length is greater than the predetermined value, displays a warning on the monitor 4. Alternatively, if the CPU 2701 determines that the converted draw length is within (Ls−Lf), then the CPU 2701 displays OK on the monitor 4. Employing (Ls−Lf−Lt) as the reference for warning or OK display may further improve the usability. The method of notification to the user is not limited to the display on the monitor 4 and may use visual information such as light emitting diode (LED), sound, or vibrations. This applies also to the following notification. In other words, the medical system 1A further includes a notification device for notifying the user whether the distance to the subject measured by the linear member 19 falls within a predetermined range. In other words, the notification device notifies the user whether the distance to the subject is longer than a predetermined length or shorter than a predetermined length.

FIG. 16C illustrates a case where the linear member 19 has a marker. In the drawing, reference sign 1601 denotes the marker. The marker may be in any discriminable color or pattern. When the linear member 19 is fully drawn out, the marker 1601 indicates the position of the distal end of the catheter 11 when the catheter apparatus 1 is attached at the second forward position or the second retracted position relative to the stage 2a, that is, the reachable position (the reference position or the target position) of the catheter 11. Setting the marker 1601 to the position corresponding to the distal end of the catheter 11 when the catheter apparatus 1 is attached to the second retracted position allows for installing the medical system 1A so that the catheter 11 may reach the target more reliably. In this case, the user does not need to compare the memorized value of the linear member 19 with the reference length displayed on the monitor 4, allowing the user to intuitively recognize suitable positioning of the medical system 1A. Setting the length to the marker 1601 when the linear member 19 is fully drawn out to the value (Ls−Lf−Lt) obtained by subtracting an average Lt or the maximum possible Lt may improve the usability. The length of the linear member 19 that can be drawn out from the support base 2 may be shorter than the distance from the support base 2 to the distal end of the catheter 11 when the catheter apparatus 1 is at the second forward position relative to the stage 2a. In the extending direction of the catheter 11, the length from the support base 2 to the distal end of the linear member 19 when the linear member 19 is fully drawn out may be shorter than the length from the support base 2 to the distal end of the catheter 11 when the catheter apparatus 1 is at the maximum forward position.

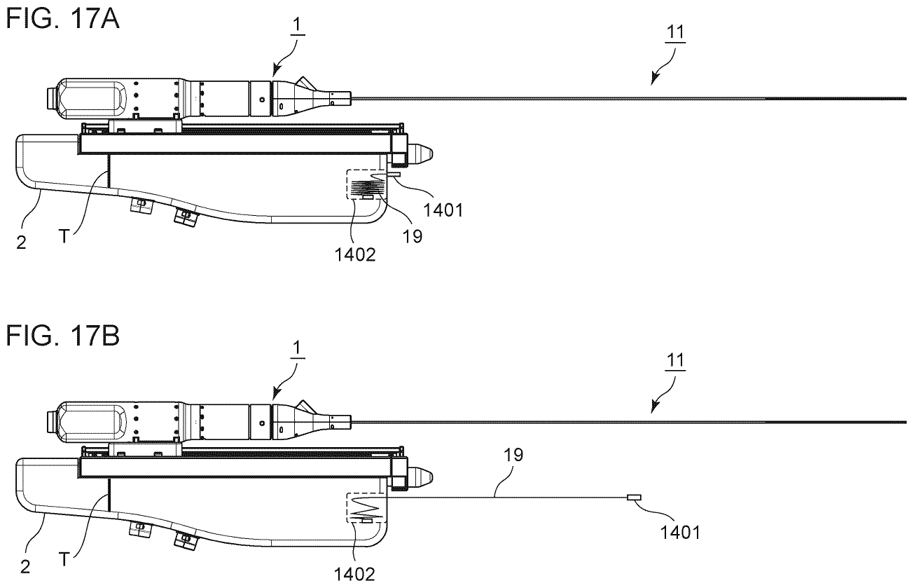

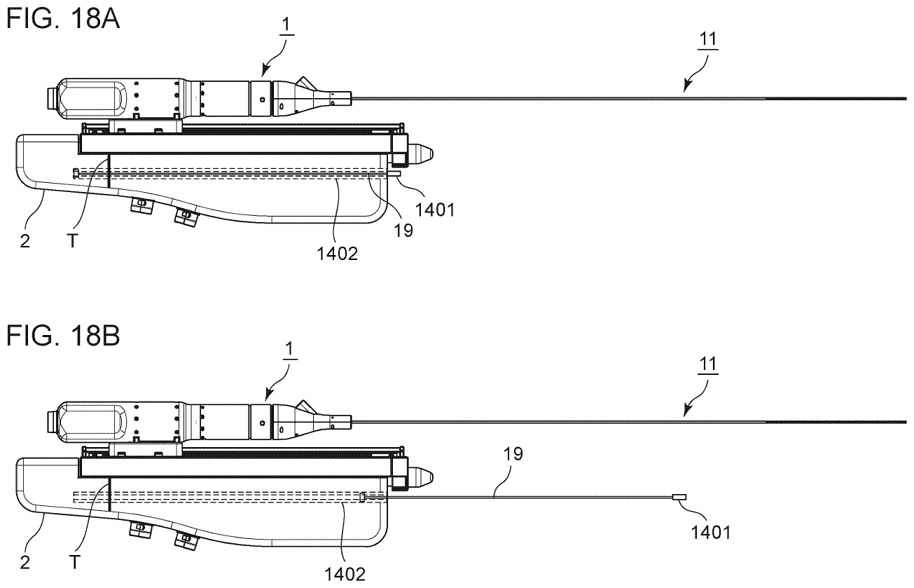

Although in the above embodiment the linear member 19 drawn out from the support base 2 is wound and housed in the support base 2, this is illustrative only. For example, the linear member 19 may be folded and housed in the support base 2, as illustrated in FIG. 17A, or may be housed in the support base 2 parallel to the X-axis, as illustrated in FIG. 18A.

As has been described above, providing the measuring device (linear member 19) for the medical system 1A allows for measuring the distance to the subject into which the catheter 11 is to be inserted. For example, this allows the user to dispose the catheter apparatus 1 of the medical system 1A properly relative to the subject by comparing the reach information displayed on the monitor 4 with the result of measurement by the measuring device and moving the cart 6. This also allow the controller 3 to display information that prompts the user to change the position of the medical system 1A on the monitor 4 or operate the notification device using the result of measurement. The monitor 4 may display information related to the target value of the distance to the subject into which the catheter 11 is to be inserted (target value information). This allows the user to dispose the medical system 1A at a suitable position relative to the subject by comparing the distance to the subject measured using the measuring device and the target value information. Both the result of measurement by the measuring device (the distance to the subject) and the target value information may be displayed on the monitor 4. The monitor 4 may display both the target value information and the reach information described in the first embodiment or one of the target value information and the reach information.

### Third Embodiment

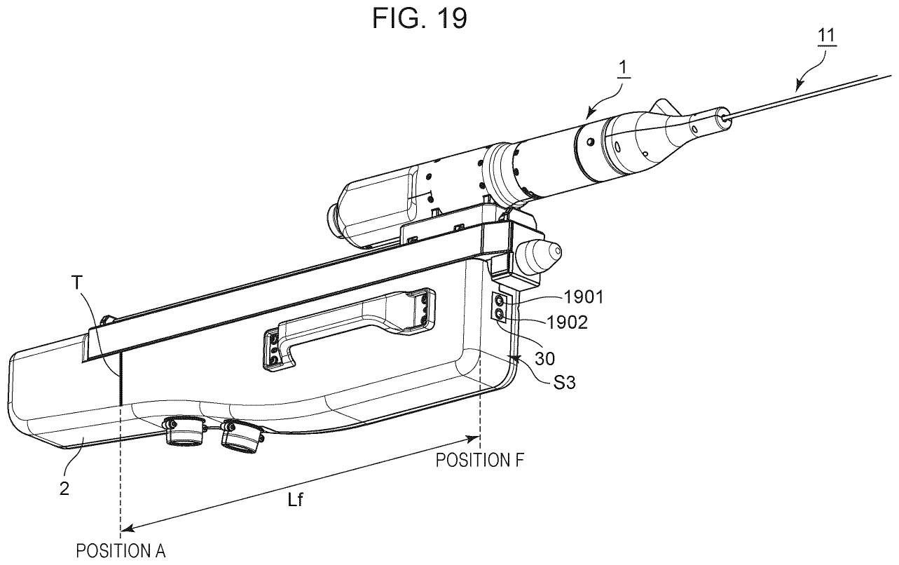

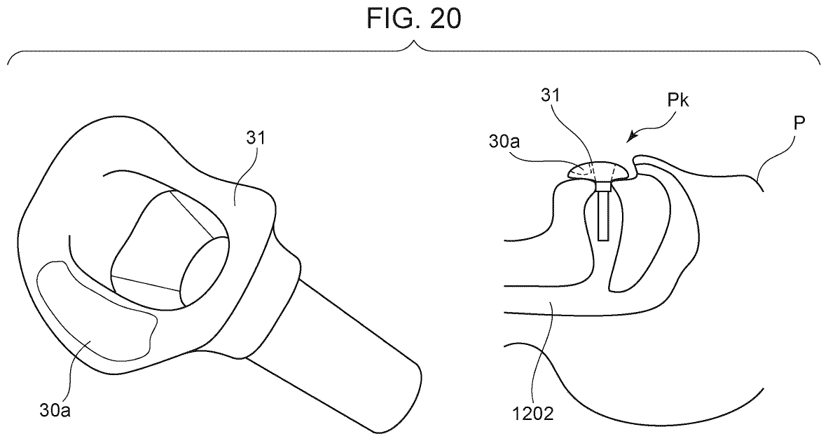

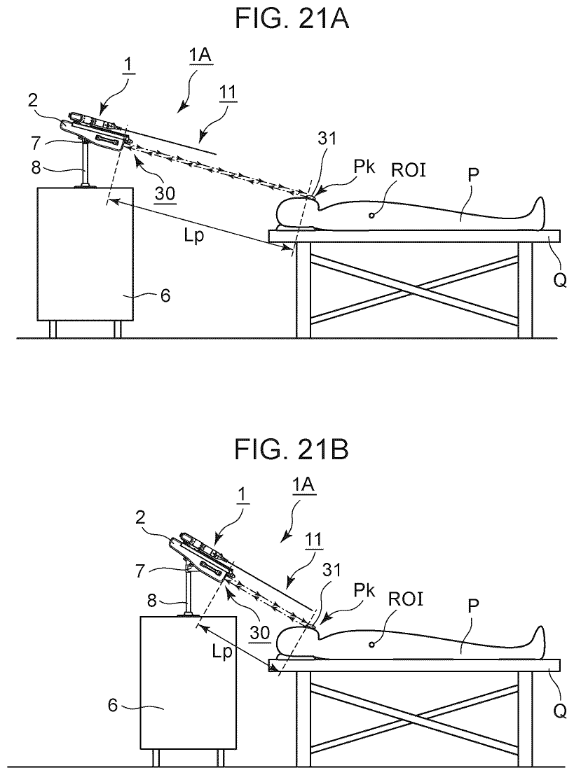

Next, a third embodiment will be described. Referring to FIGS. 19 and 20, a means for automatically measuring the separation distance between the support base 2 and the subject will be described. FIG. 19 is a perspective view of the support base 2 including a measurement sensor 30. FIG. 20 illustrates a mouthpiece 31 (detection target section) including a diffuse reflector 30a of the measurement sensor 30.

In the second embodiment, the medical system 1A includes the measuring device including the linear member 19. In this embodiment, the medical system 1A includes the measurement sensor (measuring device) 30 that measures the distance to the subject into which the catheter 11 is to be inserted using light (laser beam). As illustrated in FIG. 19, the measurement sensor 30 for measuring the separation distance from the subject is provided on a front S3 of the support base 2 in the advancing direction. The placement position of the measurement sensor 30 is designated as position F. In this embodiment, the measurement sensor 30 is a laser displacement sensor and includes a light emitting section and a light receiving section on the same surface. The measurement sensor 30 is disposed at position F, a distance Lf from the marking T. The laser beam emitted from the measurement sensor 30 is projected parallel to the advancing direction of the stage 2a. The measurement sensor 30 may be started at any timing. In this embodiment, the measurement sensor 30 (the laser displacement sensor) is started according to an instruction from the user in placing the catheter apparatus 1 at the patient's mouth but may be automatically started simultaneously with the start of the medical system 1A. The measurement sensor 30 may be stopped any timing. The controller 3 may obtain the result of measurement of the distance to the subject by the laser displacement sensor all the time while the laser displacement sensor is activated or when a user's instruction to start the measurement is issued. The measuring means is not limited to the laser displacement sensor and may be any measuring means capable of measuring the distance between two points, such as a photoelectric sensor. The CPU 2701 displays the measured distance on the monitor 4 (notification section) as visualized information based on the signal input from the measurement sensor 30. In other words, the monitor 4 displays information related to the distance to the subject measured by the measuring device 30.

As illustrated in FIG. 20, a mouthpiece with an auxiliary tube 31 for smoothly advancing the catheter 11 into the patient's lumen 1202 is used. The user attaches the mouthpiece 31 to the patient's mouth Pk while inserting the auxiliary tube into the patient's oral cavity. The mouthpiece 31 attached to the patient P includes a diffuse reflector 30a serving as a guidance mark for guiding the laser beam to the irradiation position and diffusely reflecting the laser beam. If the material of the mouthpiece 31 itself has the same surface properties as the diffuse reflector 30a, there is no need to separately provide the diffuse reflector 30a, and the diffused reflected light from the mouthpiece 31 may be detected.

The user operates the handle 18 on the support base 2 to measure the distance to the mouthpiece 31 attached to the patient located in front to adjust the position of the catheter apparatus 1 so that the laser beam is aligned with the diffuse reflector 30a. Aligning the laser beam with the diffuse reflector 30a allows for measuring the separation distance Lp between the catheter apparatus 1 and the patient's mouth Pk and linearly disposing the catheter apparatus 1 relative to the patient's mouth Pk.

The measurement sensor 30 receives the laser beam reflected by the diffuse reflector 30a to measure the separation distance Lp to the patient's mouth Pk.

At this time, the controller 3 reads a separation determination distance Lh serving as a measurement reference length from the storage device 2703. The value Lh is calculated by the controller 3 from the size of the catheter apparatus 1 and the patient-specific data obtained through planning and input to the controller 3 in advance. For example, in the case where the separation determination distance Lh is (Ls−Lf), the controller 3 compares the separation distance Lp and the separation determination distance Lh, and if the relationship Lp >Lh holds, the controller 3 determines that the relative distance between the catheter apparatus 1 and the patient is large. In this case, the controller 3 notifies the user of the fact using, for example, the monitor 4 (notification section). The user checks the display on the monitor 4 and adjusts the position of the catheter apparatus 1 in the direction in which the relative distance between the catheter apparatus 1 and the patient reaches the relationship Lp≤Lh. In contrast, if the controller 3 compares the separation distance Lp and the separation determination distance Lh and determines that the relationship Lp≤Lh holds, then the controller 3 determines that the relative distance between the catheter apparatus 1 and the patient has reached the distance at which the distal end of the catheter 11 reaches the ROI and notifies the user of the fact using, for example, the monitor 4. The user checks the display on the monitor 4 and completes the adjustment of the installation of the catheter apparatus 1. Also in the case where the separation determination distance Lh is set at (Le−Lf) or (Ls−Lf−Lt), the controller 3 may make a similar determination. Furthermore, since the controller 3 displays the separation determination distance Lh in the notification area 1301 of the monitor 4, the usability for the user is improved.

In this case, a message to prompt the user to bring the catheter apparatus 1 close to the patient (for example, a message that the distal end of the catheter 11 cannot reach the ROI) or a message that the positioning has been completed (for example, a message indicating that the distal end of the catheter 11 can be guided to the ROI) may be additionally displayed. A placement image of the catheter apparatus 1 may be displayed on the monitor 4. The notification to user may be visual information such as an LED, sound, or vibrations.

The controller 3 may display the sum of the measured value from the measurement sensor 30 and Lf on the monitor 4. The controller 3 displays this value together with the reach length information 1302, for example, in the notification area 1301 of the monitor 4 described in the first embodiment. This allows the user to compare the measured value with the reach length information 1302, improving the usability in adjusting the positioning of the catheter apparatus 1.

Thus, since the medical system 1A includes the measuring device (measurement sensor 30), the distance to the subject into which the catheter 11 is to be inserted may be measured. For example, the user may properly dispose the catheter apparatus 1 of the medical system 1A relative to the subject by comparing the reach information displayed on the monitor 4 with the result of measurement by the measurement sensor 30 and moving the cart 6. The controller 3 may display information that prompts the user to change the positioning of the medical system 1A on the monitor 4 or operate the notification device using the result of measurement. Information related to the target value of the distance to the subject into which the catheter 11 is to be inserted (target value information) may be displayed on the monitor 4. This allows the user to dispose the medical system 1A at a proper position relative to the subject by comparing the distance to the subject measured using the measurement sensor 30 with the target value information. Both the result of measurement by the measurement sensor 30 (the distance to the subject) and the target value information may be displayed on the monitor 4. Both or one of the target value information and the reach information described in the first embodiment may be displayed on the monitor 4.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

The present invention may support the medical system positioning operation for the patient, thereby reducing the load on the healthcare professional.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A medical system comprising:
a catheter apparatus including a bendable member having a distal end;
a receiving section to which the catheter apparatus is attached;
a support base that supports the receiving section to move linearly between a forward position and a retracted position;
a cart connected to the support base, the cart being configured to move the catheter apparatus; and
a display configured to display reach information related to a position of the distal end.

2. The medical system according to claim 1,
wherein the catheter apparatus is attachable to the receiving section at a leading end position and a trailing end position, and
wherein, in an extending direction of the bendable member, a distance between the distal end and the support base is longer when the catheter apparatus is at the leading end position than when the catheter apparatus is at the trailing end position.

3. The medical system according to claim 2, wherein the reach information includes information related to a position of the distal end when the catheter apparatus is at the leading end position.

4. The medical system according to claim 1, wherein the reach information includes information related to a length from part of the support base to the distal end.

5. The medical system according to claim 4, wherein the display displays relationship between the part of the support base and the reach information.

6. The medical system according to claim 1, wherein the display displays relationship between a subject into which the bendable member is to be inserted and the reach information.

7. The medical system according to claim 1, wherein the display prompts a user to change positioning of the catheter apparatus relative to the subject into which the bendable member is to be inserted.

8. The medical system according to claim 1, further comprising a measuring device configured to measure a distance to the subject into which the bendable member is to be inserted.

9. The medical system according to claim 8, wherein the measuring device includes a linear member configured to be drawn out from the support base.

10. The medical system according to claim 8, wherein the measuring device measures the distance using light.

11. The medical system according to claim 8, wherein the display displays information related to the distance measured by the measuring device.

12. The medical system according to claim 8, further comprising a notification device configured to notify a user whether the distance measured by the measuring device falls within a predetermined range.

13. The medical system according to claim 1, further comprising a linear member configured to be drawn out from the support base.

14. A medical system comprising:
a catheter apparatus including a bendable member having a distal end;
a receiving section to which the catheter apparatus is attached;
a support base that supports the receiving section to move linearly between a forward position and a retracted position;
a cart connected to the support base, the cart being configured to move the catheter apparatus; and
a measuring device configured to measure a distance to a subject into which the bendable member is to be inserted.

15. The medical system according to claim 14,
wherein the catheter apparatus is attachable to the receiving section at a leading end position and a trailing end position, and
wherein a distance between the distal end and the support base is longer when the catheter apparatus is at the leading end position than when the catheter apparatus is at the trailing end position.

16. The medical system according to claim 14, wherein the measuring device includes a linear member configured to be drawn out from the support base.

17. The medical system according to claim 14, wherein the measuring device measures the distance using light.

18. The medical system according to claim 14, further comprising a notification device configured to notify a user whether the distance measured by the measuring device falls within a predetermined range.

19. The medical system according to claim 14, further comprising a display configured to display information.

20. The medical system according to claim 19, wherein the display displays information related to the distance measure by the measuring device.

21. The medical system according to claim 19, wherein the display displays information related to a target value of the distance.

22. The medical system according to claim 19, wherein the display prompts the user to change positioning of the catheter apparatus relative to the subject.

23. A medical system comprising:
a catheter apparatus including a bendable member having a distal end;
a receiving section to which the catheter apparatus is attached;
a support base that supports the receiving section to move linearly between a forward position and a retracted position;
a cart connected to the support base, the cart being configured to move the catheter apparatus; and
a display configured to display information related to a target value of a distance to a subject into which the bendable member is to be inserted.

24. The medical system according to claim 23,
wherein the catheter apparatus is attachable to the receiving section at a leading end position and a trailing end position, and
wherein, in an extending direction of the bendable member, a distance between the distal end and the support base is longer when the catheter apparatus is at the leading end position than when the catheter apparatus is at the trailing end position.

25. The medical system according to claim 23, further comprising a measuring device configured to measure a distance to the subject into which the bendable member is to be inserted.

26. The medical system according to claim 25, wherein the measuring device includes a linear member configured to be drawn out from the support base.

27. The medical system according to claim 25, wherein the measuring device measures the distance using light.

28. The medical system according to claim 25, wherein the display displays information related to the distance measured by the measuring device.

29. The medical system according to claim 25, further comprising a notification device configured to notify a user whether the distance measured by the measuring device falls within a predetermined range.

30. A medical system comprising:
a catheter apparatus including a bendable member having a distal end;
a receiving section to which the catheter apparatus is attached;
a support base that supports the receiving section to move linearly between a forward position and a retracted position;
a cart connected to the support base, the cart being configured to move the catheter apparatus; and
a linear member configured to be drawn out from the support base.

31. The medical system according to claim 30,
wherein the catheter apparatus is attachable to the receiving section at a leading end position and a trailing end position, and
wherein, in an extending direction of the bendable member, a distance between the distal end and the support base is longer when the catheter apparatus is at the leading end position than when the catheter apparatus is at the trailing end position.

32. The medical system according to claim 31, wherein a length of the linear member drawable from the support base is shorter than the distance from the support base to the distal end when the catheter apparatus is at the leading end position.

33. The medical system according to claim 1, wherein the catheter apparatus is detachable from the receiving section.
